(19) United States
(12) Patent Application Publication
(10) Pub. No.: US 2023/0140925 A1
(43) Pub. Date: May 11, 2023

(54) COMPOUND FOR ORGANIC ELECTRICAL ELEMENT, ORGANIC ELECTRICAL ELEMENT USING SAME, AND ELECTRONIC DEVICE COMPRISING SAME

(71) Applicant: DUK SAN NEOLUX CO., LTD., Cheonan-si, Chungcheongnam-do (KR)

(72) Inventors: Hyoung Keun PARK, Cheonan-si, Chungcheongnam-do (KR); Hyun Ji OH, Cheonan-si, Chungcheongnam-do (KR); Dae Sic KIM, Cheonan-si, Chungcheongnam-do (KR); Sun Hee LEE, Cheonan-si, Chungcheongnam-do (KR); Soung Yun MUN, Cheonan-si, Chungcheongnam-do (KR); Bum Sung LEE, Cheonan-si, Chungcheongnam-do (KR); Nam Kyun KIM, Cheonan-si, Chungcheongnam-do (KR)

(73) Assignee: DUK SAN NEOLUX CO., LTD., Cheonan-si, Chungcheongnam-do (KR)

(21) Appl. No.: 17/995,714

(22) PCT Filed: Apr. 8, 2021
(86) PCT No.: PCT/KR2021/004435

§ 371 Date: Oct. 7, 2022

(30) Foreign Application Priority Data
Apr. 10, 2020 (KR) 10-2020-0044009

(51) Int. Cl. H10K 85/60; C07C 211/61; C09K 11/06; C07D 307/91

(52) U.S. Cl. CPC H01L 51/006; C07C 211/61; C09K 11/06; C07D 307/91; H01L 51/0061; H01L 51/5012

(57) ABSTRACT

Provided are a compound capable of improving the light-emitting efficiency, stability, and lifespan of an element; an organic electrical element using same; and an electronic device comprising same.

### BACKGROUND

### Technical Field

The present invention relates to a compound for an organic electric element, an organic electric element using same, and an electronic device thereof.

### Background Art

The flat panel display plays a very important role in supporting the advanced image information society based on the Internet, which is showing rapid growth in recent years. In particular, an organic electroluminescent device (organic EL device) capable of low-voltage driving as a self-emission type has superior viewing angles and contrast ratios compared to liquid crystal displays (LCDs), which are the mainstream of flat panel display devices, and does not require a backlight, so it can be lightweight and thin, and has advantages in terms of power consumption. In addition, it is attracting attention as a next-generation display device because of its fast response speed and wide color reproduction range. In general, an organic EL device is formed on a glass substrate in the order of an anode made of a transparent electrode, an organic thin film including a light emitting region, and a metal electrode (cathode). In this case, the organic thin film comprises a hole injection layer (HIL), a hole transport layer (HTL), an electron transport layer (ETL) or an electron injection layer(HIL) in addition to an emitting layer (EML) and further include an electron blocking layer (EBL) or a hole blocking layer (HBL), and an emitting auxiliary layer due to the light emitting characteristics of the emitting layer. When an electric field is applied to the organic EL device having such a structure, holes are injected from the anode and electrons are injected from the cathode, and the injected holes and electrons pass through the hole transport layer and the electron transport layer, respectively, and recombine in the emitting layer to form light emitting excitons. The formed luminescent excitons emit light while transitioning to ground states, at this time, in order to increase the efficiency and stability of the light emitting state, a light emitting dye (guest) is also doped into the emitting layer (host). In order to utilize such an organic electronic device in various display media, the lifespan of the element is more important than anything else, and various studies are being conducted to increase the lifespan of the organic electric element. In particular, various studies are being conducted on organic materials inserted into a buffer layer such as a hole transport layer or an emitting auxiliary layer for excellent lifespan characteristics of organic electric devices, and to this end, a material for a hole injection layer and a hole transport layer with high uniformity and low crystallinity when forming a thin film after deposition while providing high hole transport properties from the anode to the organic layer is required.

It is necessary to develop a material for a hole injection layer and a hole transport layer not only having stable characteristics against Joule heating generated during device driving, that is, a high glass transition temperature, but also delaying the penetration and diffusion of metal oxide from the anode electrode (ITO), which is one of the causes of shortening the lifespan of organic electric devices. In addition, it is reported that the low glass transition temperature of the hole transport layer material greatly affects the device lifespan according to the characteristic that the uniformity of the thin film surface is collapsed during device driving. In addition, in the formation of OLED devices, the deposition method is the mainstream, and a material that can withstand this deposition method for a long time, that is, a material with strong heat resistance characteristics is required.

In particular, it is urgent to overcome the problems of power consumption and lifespan, as the main overcoming challenges of organic light emitting diodes are enlarged in panel sizes of mobile phones and tablet PCs.

However, as a hole transport layer material, it is difficult to overcome the driving voltage and lifetime at the same time. The reason is that, in order to lower the driving voltage, materials with excellent hole transport ability, ie, high hole mobility, have a planar structure rich in electrons in most cases. For example, naphthyl, fluorene and phenanthrene and the like.

However, when a compound of the above structure is introduced into a hole transport material as a substituent, the hole mobility increases up to a certain number and has a good effect on the lifespan, but if the number of introductions in the molecule is increased to reach the low voltage driving target required in the current industry, the driving voltage is lowered and low voltage driving is possible, but the lifespan characteristics are rapidly deteriorated.

The reason for this is that, in the case of molecules in which electron-rich planar structures are excessively introduced, holes are trapped between the plate-like structures and stabilized when a constant current is continuously supplied during device lifespan evaluation, this lowers the hole mobility and eventually increases the driving voltage to apply a constant current, resulting in a sharp deterioration in device lifespan. It is expressed by the following formula.

$$J = \frac{9}{8} εμ \frac{{V}^{2}}{{d}^{3}} \\ θ = \frac{9}{8} εμ \frac{1}{d} {F}^{2} θ$$

(J=Space Charge limited current, ϵ=Permissibility, μ=Mobility Coefficient, θ=Charge Trap Coefficient (Free Carrier/Total Carrier), V=Voltage, d=Thickness) As the number of free carriers decreases due to the trap phenomenon, the 8 value decreases, therefore, in the current-driven organic light emitting device that requires a constant current, the driving voltage is increased, which can have a very fatal result on the lifespan. Therefore, as described above, the introduction of an electron-rich plate-like structure capable of increasing hole mobility over a certain amount adversely affects the lifespan, so the possibility of lowering the driving voltage by using it is not great.

Accordingly, the present inventors confirmed that the compound substituted with deuterium showed many thermodynamic behaviors compared to the unsubstituted compound, and that, among these thermodynamic properties, when the iridium compound is substituted with deuterium, the properties vary according to the difference in carbon, hydrogen, and carbon and deuterium bond lengths, and that the compound composed of deuterium can have higher luminous efficiency due to the weakening of the intermolecular van der Waals force generated by the shorter bond length compared to the compound not substituted with deuterium.

However, a method of lowering the driving voltage by substituting with deuterium, that is, increasing the hole transport Mobility of the hole transport material, has not been studied much at present, and the prior art that demonstrates the effect according to a specific deuterium substitution rate has not yet been reported. Moreover, the conventionally reported general deuterium substitution method has a disadvantage in that it is difficult to control the substitution rate.

### DETAILED DESCRIPTION OF THE INVENTION

### Summary

For the purpose of solving the problems of the above-mentioned background art, in the present invention, deuterium is substituted in a specific ratio of 59% to 73% to an amine-based compound having long lifespan, thereby completing a device with a long lifespan, in order to realize a long-life device, which is a required characteristic of an organic electric device.

Accordingly, an object of the present invention is to provide a compound deuterated at a specific ratio, an organic electric element using same, and an electronic device thereof.

### Technical Solution

The present invention provides a 59% to 73% deuterated compound represented by Formula 1.

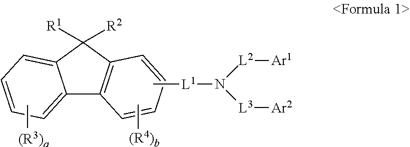

In another aspect, the present invention provides a method for preparing the 59% to 73% deuterated compound represented by Formula 1.

In another aspect, the present invention provides an organic electric element and an electronic device comprising the compound represented by Formula 1.

### Effects of the Invention

By using the compound according to the present invention, high luminous efficiency, low driving voltage and high heat resistance of the element can be achieved, and color purity and lifespan of the element can be greatly improved.

### BRIEF DESCRIPTION OF THE DRAWINGS

FIG. 1 to FIG. 3 are exemplary views of an organic electroluminescent device according to the present invention.

FIG. 4 shows a Formula according to an aspect of the present invention.

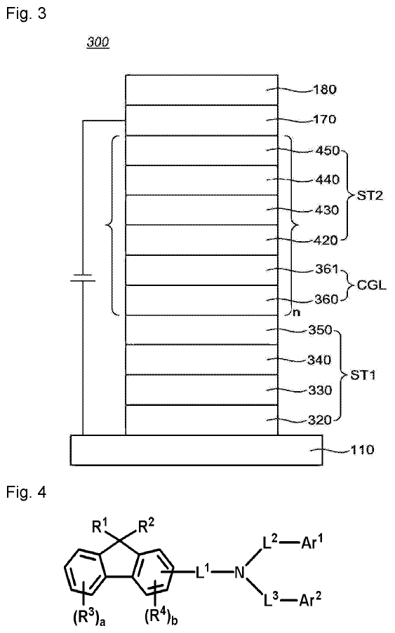

100, 200, 300: organic electronic element 110: the first electrode 120: hole injection layer 130: hole transport layer 140: emitting layer 150: electron transport layer 160: electron injection layer 170: second electrode 160: electron transport layer 170: electron injection layer 180: light efficiency enhancing Layer 210: buffer layer 220: emitting auxiliary layer 320: first hole injection layer 330: first hole transport layer 340: first emitting layer 350: first electron transport layer 360: first charge generation layer 361: second charge generation layer 420: second hole injection layer 430: second hole transport layer 440: second emitting layer 450: second electron transport layer CGL: charge generation layer ST1: first stack ST2: second stack

### DETAILED DESCRIPTION

Hereinafter, some embodiments of the present invention will be described in detail. Further, in the following description of the present invention, a detailed description of known functions and configurations incorporated herein will be omitted when it may make the subject matter of the present invention rather unclear.

In addition, terms, such as first, second, A, B, (a), (b) or the like may be used herein when describing components of the present invention. Each of these terminologies is not used to define an essence, order or sequence of a corresponding component but used merely to distinguish the corresponding component from other component(s). It should be noted that if a component is described as being “connected”, “coupled”, or “connected” to another component, the component may be directly connected or connected to the other component, but another component may be “connected”, “coupled” or “connected” between each component.

As used in the specification and the accompanying claims, unless otherwise stated, the following is the meaning of the term as follows.

Unless otherwise stated, the term “halo” or “halogen”, as used herein, includes fluorine, bromine, chlorine, or iodine.

Unless otherwise stated, the term “alkyl” or “alkyl group”, as used herein, has a single bond of 1 to 60 carbon atoms, and means saturated aliphatic functional radicals including a linear alkyl group, a branched chain alkyl group, a cycloalkyl group (alicyclic), an cycloalkyl group substituted with a alkyl or an alkyl group substituted with a cycloalkyl.

Unless otherwise stated, the term “alkenyl” or “alkynyl”, as used herein, has double or triple bonds of 2 to 60 carbon atoms, but is not limited thereto, and includes a linear or a branched chain group.

Unless otherwise stated, the term “cycloalkyl”, as used herein, means alkyl forming a ring having 3 to 60 carbon atoms, but is not limited thereto.

Unless otherwise stated, the term “alkoxyl group”, “alkoxy group” or “alkyloxy group”, as used herein, means an oxygen radical attached to an alkyl group, but is not limited thereto, and has 1 to 60 carbon atoms.

Unless otherwise stated, the term “aryloxyl group” or “aryloxy group”, as used herein, means an oxygen radical attached to an aryl group, but is not limited thereto, and has 6 to 60 carbon atoms.

The terms “aryl group” and “arylene group” used in the present invention have 6 to 60 carbon atoms, respectively, unless otherwise specified, but are not limited thereto. In the present invention, an aryl group or an arylene group means a single ring or multiple ring aromatic, and includes an aromatic ring formed by an adjacent substituent joining or participating in a reaction.

For example, the aryl group may be a phenyl group, a biphenyl group, a fluorene group, or a spirofluorene group.

The prefix “aryl” or “ar” means a radical substituted with an aryl group. For example, an arylalkyl may be an alkyl substituted with an aryl, and an arylalkenyl may be an alkenyl substituted with aryl, and a radical substituted with an aryl has a number of carbon atoms as defined herein.

Also, when prefixes are named subsequently, it means that substituents are listed in the order described first. For example, an arylalkoxy means an alkoxy substituted with an aryl, an alkoxylcarbonyl means a carbonyl substituted with an alkoxyl, and an arylcarbonylalkenyl also means an alkenyl substituted with an arylcarbonyl, wherein the arylcarbonyl may be a carbonyl substituted with an aryl.

Unless otherwise stated, the term “heterocyclic group”, as used herein, contains one or more heteroatoms, but is not limited thereto, has 2 to 60 carbon atoms, includes any one of a single ring or multiple ring, and may include heteroaliphadic ring and heteroaromatic ring. Also, the heterocyclic group may also be formed in conjunction with an adjacent group.

Unless otherwise stated, the term “heteroatom”, as used herein, represents at least one of N, O, S, P, or Si.

Also, the term “heterocyclic group” may include a ring including SO₂ instead of carbon consisting of cycle. For example, “heterocyclic group” includes the following compound.

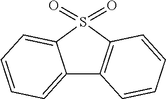 [SMILES: O=S1(=O)c2ccccc2-c2ccccc21]

Unless otherwise stated, the term “fluorenyl group” or “fluorenylene group”, as used herein, means a monovalent or divalent functional group, in which R, R′ and R″ are all hydrogen in the following structures, and the term “substituted fluorenyl group” or “substituted fluorenylene group” means that at least one of the substituents R, R′, R″ is a substituent other than hydrogen, and include those in which R and R′ are bonded to each other to form a spiro compound together with the carbon to which they are bonded.

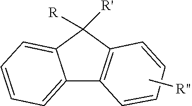

The term “spiro compound”, as used herein, has a ‘spiro union’, and a spiro union means a connection in which two rings share only one atom. At this time, atoms shared in the two rings are called ‘spiro atoms’, and these compounds are called ‘monospiro-’, ‘di-spiro ’ and ‘trispiro-’, respectively, depending on the number of spiro atoms in a compound.

Unless otherwise stated, the term “aliphatic”, as used herein, means an aliphatic hydrocarbon having 1 to 60 carbon atoms, and the term “aliphatic ring”, as used herein, means an aliphatic hydrocarbon ring having 3 to 60 carbon atoms.

Unless otherwise stated, the term “ring”, as used herein, means an aliphatic ring having 3 to 60 carbon atoms, or an aromatic ring having 6 to 60 carbon atoms, or a hetero ring having 2 to 60 carbon atoms, or a fused ring formed by the combination of them, and includes a saturated or unsaturated ring.

Other hetero compounds or hetero radicals other than the above-mentioned hetero compounds include, but are not limited thereto, one or more heteroatoms.

Also, unless expressly stated, as used herein, “substituted” in the term “substituted or unsubstituted” means substituted with one or more substituents selected from the group consisting of deuterium, halogen, an amino group, a nitrile group, a nitro group, a C₁-C₂₀ alkyl group, a C₁-C₂₀ alkoxyl group, a C₁-C₂₀ alkylamine group, a C₁-C₂₀ alkylthiopen group, a C₆-C₂₀ arylthiopen group, a C₂-C₂₀ alkenyl group, a C₂-C₂₀ alkynyl group, a C₃-C₂₀ cycloalkyl group, a C₆-C₂₀ aryl group, a C₆-C₂₀ aryl group substituted by deuterium, a C₈-C₂₀ arylalkenyl group, a silane group, a boron group, a germanium group, and a C₂-C₂₀ heterocyclic group, but is not limited to these substituents.

Also, unless there is an explicit explanation, the formula used in the present invention is the same as the definition of the substituent by the exponent definition of the following formula.

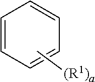

Here, when a is an integer of 0, the substituent R¹ is absent, when a is an integer of 1, the sole substituent R¹ is linked to any one of the carbon constituting the benzene ring, when a is an integer of 2 or 3, each is bonded as follows, where R¹ may be the same or different from each other, when a is an integer of 4 to 6, it is bonded to the carbon of the benzene ring in a similar manner, while the indication of the hydrogen bonded to the carbon forming the benzene ring is omitted.

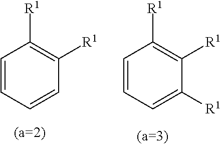

As used herein, the term “deuterated” refers to a compound or group in which deuterium is present at 100 times or more of its natural abundance level.

As used herein, the term “perdeuterated” refers to a compound or group in which all hydrogens have been replaced with deuterium. The term deuterated is synonymous with “100% deuterated”.

As used herein, the term “deutero-acid” refers to a compound capable of ionizing to donate deuterium ions to Bronsted's base. As used herein, deuterium-acids do not contain ionizable hydrogens.

Hereinafter, a compound according to an aspect of the present invention and an organic electric element including the same will be described.

The present invention is a method that can lower the driving voltage without introducing a plate-shaped molecular structure that adversely affects the lifespan by using a material with a good lifespan, and by using a method of substituting deuterium at an appropriate ratio, provides a method of lowering the driving voltage.

When substituted with deuterium, the zero point energy, that is, the energy of the ground state is lowered, and as the bond length of deuterium-carbon becomes shorter than that of hydrogen-carbon, the molecular hardcore volume decreases, accordingly, electrical polarizability can be reduced, by weakening the intermolecular interaction, the thin film volume can be increased. This property can lower the crystallinity of the thin film, which is, create an amorphous state, and is generally very effective in implementing an amorphous state, which is essential to increase OLED lifespan and driving characteristics.

In addition, when a film is formed with a compound substituted with deuterium, the film is formed in an amorphous glass state that can greatly affect the hole mobility of the thin film, and this amorphous glass state can reduce the grain boundary through isotropic and homogeneous properties, thereby speeding up the flow of charges, that is, hole mobility.

The present invention provides a 59% to 73% deuterated compound represented by Formula 1.

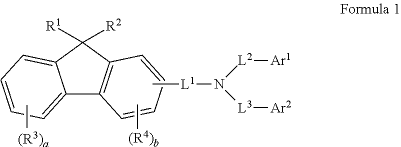

Wherein:

1) R¹ and R² are each independently a C₁-C₂₀ alkyl group, and R¹ and R² cannot be bonded to each other to form a ring;

Wherein R¹ and R² are preferably a C₁-C₃₀ alkyl group, more preferably a C₁-C₂₄ alkyl group,

2) R³ and R⁴ are each independently the same as or different from each other, and each independently selected from the group consisting of hydrogen; deuterium; halogen; cyano group; nitro group; a C₆-C₆₀ aryl group; fluorenyl group; a C₂-C₆₀ heterocyclic group including at least one heteroatom of O, N, S, Si or P; a fused ring group of a C₃-C₆₀ aliphatic ring and a C₆-C₆₀ aromatic ring; a C₁-C₅₀ alkyl group; a C₂-C₂₀ alkenyl group; a C₂-C₂₀ alkynyl group; a C₁-C₃₀ alkoxyl group; a C₆-C₃₀ aryloxy group; and -L′-N(Rₐ)(R_{b}); or, when a and b are 2 or more, a plurality of adjacent R³ or a plurality of R⁴ may be bonded to each other to form a ring, Wherein R³ and R⁴ are an aryl group, they may be preferably a C₆-C₃₀ aryl group, more preferably a C₆-C₂₅ aryl group, for example, phenylene, biphenyl, naphthalene, terphenyl, etc.

Wherein in case R³ and R⁴ are a heterocyclic group, they may be preferably a C₂-C₃₀ heterocyclic group, more preferably a C₂˜C₂₄ heterocyclic group, for example, pyrazine, thiophene, pyridine, pyrimidoindole, 5-phenyl-5H-pyrimido[5,4-b]indole, quinazoline, benzoquinazoline, carbazole, dibenzoquinazoline, dibenzofuran, dibenzothiophen, benzothienopyrimidine, benzofuropyrimidine, phenothiazine, phenylphenothiazine, etc.

Wherein in case R³ and R⁴ are a fused ring groups, they may be preferably a fused ring group of a C₃-C₃₀ aliphatic ring and a C₆-C₃₀ aromatic ring, more preferably a fused ring group of a C₃-C₂₄ aliphatic ring and a C₆-C₂₄ aromatic ring.

Wherein in case R³ and R⁴ are an alkyl group, they may be preferably a C₁-C₃₀ alkyl group, more preferably a C₃-C₂₄ alkyl group.

Wherein in case R³ and R⁴ are an alkoxyl group, they may be preferably an C₁˜C₂₄ alkoxyl group.

Wherein in case R³ and R⁴ are an aryloxy group, they may be preferably an C₆˜C₂₄ aryloxy group.

Wherein L′ is selected from the group consisting of a single bond; a C₆-C₆₀ arylene group; fluorenylene group; a fused ring group of a C₃-C₆₀ aliphatic ring and a C₆-C₆₀ aromatic ring; a C₂-C₆₀ heterocyclic group; wherein Rₐ and R_{b} are each independently selected from the group consisting of a C₆-C₆₀ aryl group; fluorenyl group; a fused ring group of a C₃-C₆₀ aliphatic ring and a C₆-C₆₀ aromatic ring; a C₂-C₆₀ heterocyclic group including at least one heteroatom of O, N, S, Si or P;

Wherein in case L′ is an arylene group, it may be preferably a C₆-C₃₀ arylene group, more preferably a C₆-C₂₅ arylene group, for example, phenylene, biphenyl, naphthalene, terphenyl, etc.

Wherein in case L′ is a heterocyclic group, it may be preferably a C₂˜C₃₀ heterocyclic group, more preferably a C₂˜C₂₄ heterocyclic group, for example, pyrazine, thiophene, pyridine, pyrimidoindole, 5-phenyl-5H-pyrimido[5,4-b]indole, quinazoline, benzoquinazoline, carbazole, dibenzoquinazoline, dibenzofuran, benzothienopyrimidine, benzofuropyrimidine, phenothiazine, phenylphenothiazine, etc.

Wherein in case L′ is a fused ring groups, it may be preferably a fused ring group of a C₃-C₃₀ aliphatic ring and a C₆-C₃₀ aromatic ring, more preferably a fused ring group of a C₃-C₂₄ aliphatic ring and a C₆-C₂₄ aromatic ring.

Wherein in case Rₐ and R_{b} are an aryl group, they may be preferably a C₆-C₃₀ aryl group, more preferably a C₆-C₂₅ aryl group, for example, phenylene, biphenyl, naphthalene, terphenyl, etc.

Wherein in case Rₐ and R_{b} are a fused ring groups, they may be preferably a fused ring group of a C₃-C₃₀ aliphatic ring and a C₆-C₃₀ aromatic ring, more preferably a fused ring group of a C₃-C₂₄ aliphatic ring and a C₆-C₂₄ aromatic ring.

Wherein in case Rₐ and R_{b} are a heterocyclic group, they may be preferably a C₂˜C₃₀ heterocyclic group, more preferably a C₂˜C₂₄ heterocyclic group, for example, pyrazine, thiophene, pyridine, pyrimidoindole, 5-phenyl-5H-pyrimido[5,4-b]indole, quinazoline, benzoquinazoline, carbazole, dibenzoquinazoline, dibenzofuran, benzothienopyrimidine, benzofuropyrimidine, phenothiazine, phenylphenothiazine, etc.

L¹, L² and L³ are each independently selected from the group consisting of a single bond; a C₆-C₆₀ arylene group; fluorenylene group; a fused ring group of a C₃-C₆₀ aliphatic ring and a C₆-C₆₀ aromatic ring; and a C₂-C₆₀ heterocyclic group;

Wherein in case L¹, L² and L³ are an arylene group, they may be preferably a C₆-C₃₀ arylene group, more preferably a C₆-C₂₅ arylene group, for example, phenylene, biphenyl, naphthalene, terphenyl, etc.

Wherein in case L¹, L² and L³ are a heterocyclic group, they may be preferably a C₂˜C₃₀ heterocyclic group, more preferably a C₂˜C₂₄ heterocyclic group, for example, pyrazine, thiophene, pyridine, pyrimidoindole, 5-phenyl-5H-pyrimido[5,4-b]indole, quinazoline, benzoquinazoline, carbazole, dibenzoquinazoline, dibenzofuran, benzothienopyrimidine, benzofuropyrimidine, phenothiazine, phenylphenothiazine, etc.

Wherein in case L¹, L² and L³ are a fused ring groups, they may be preferably a fused ring group of a C₃-C₃₀ aliphatic ring and a C₆-C₃₀ aromatic ring, more preferably a fused ring group of a C₃-C₂₄ aliphatic ring and a C₆-C₂₄ aromatic ring.

4) a is an integer of 0 to 4, b is an integer of 0 to 3,

5) Ar¹ and Ar² are each independently selected from the group consisting of a C₆-C₆₀ aryl group; a C₂-C₆₀ heterocyclic group including at least one heteroatom of O, N, S, Si or P; a fused ring group of a C₃-C₆₀ aliphatic ring and a C₆-C₆₀ aromatic ring; a C₁-C₆₀ alkyl group; a C₂-C₂₀ alkenyl group; a C₂-C₂₀ alkynyl group; a C₁-C₃₀ alkoxyl group; a C₆-C₃₀ aryloxy group; and -L′-N(Rₐ)(R_{b});

Wherein in case Ar¹ and Ar² are an aryl group, they may be preferably a C₆-C₃₀ aryl group, more preferably a C₆-C₂₅ aryl group, for example, phenylene, biphenyl, naphthalene, terphenyl, etc.

Wherein in case Ar¹ and Ar² are a heterocyclic group, they may be preferably a C₂˜C₃₀ heterocyclic group, more preferably a C₂˜C₂₄ heterocyclic group, for example, pyrazine, thiophene, pyridine, pyrimidoindole, 5-phenyl-5H-pyrimido[5,4-b]indole, quinazoline, benzoquinazoline, carbazole, dibenzoquinazoline, dibenzofuran, benzothienopyrimidine, benzofuropyrimidine, phenothiazine, phenylphenothiazine, etc.

Wherein in case Ar¹ and Ar² are a fused ring groups, they may be preferably a fused ring group of a C₃-C₃₀ aliphatic ring and a C₆-C₃₀ aromatic ring, more preferably a fused ring group of a C₃-C₂₄ aliphatic ring and a C₆-C₂₄ aromatic ring.

Wherein in case Ar¹ and Ar² are an alkyl group, they may be preferably a C₁-C₃₀ alkyl group, more preferably a C₁-C₂₄ alkyl group. Wherein in case Ar¹ and Ar² are an alkoxyl group, they may be preferably an C₁˜C₂₄ alkoxyl group.

Wherein in case Ar¹ and Ar² are an aryloxy group, they may be preferably an C₆˜C₂₄ aryloxy group.

6) wherein the aryl group, arylene group, heterocyclic group, fluorenyl group, fluorenylene group, fused ring group, alkyl group, alkenyl group, alkoxy group and aryloxy group may be substituted with one or more substituents selected from the group consisting of deuterium; halogen; silane group; siloxane group; boron group; germanium group; cyano group; nitro group; C₁-C₂₀ alkylthio group; C₁-C₂₀ alkoxyl group; C₁-C₂₀ alkyl group; C₂-C₂₀ alkenyl group; C₂-C₂₀ alkynyl group; C₆-C₂₀ aryl group; C₆-C₂₀ aryl group substituted with deuterium; a fluorenyl group; C₂˜-C₂₀ heterocyclic group; C₃-C₂₀ cycloalkyl group; C₇-C₂₀ arylalkyl group; and C₈-C₂₀ arylalkenyl group; and and -L′-N(Rₐ)(R_{b}); also the substituents may be bonded to each other to form a saturated or unsaturated ring, wherein the term ‘ring’ means a C₃-C₆₀ aliphatic ring or a C₆-C₆₀ aromatic ring or a C₂-C₆₀ heterocyclic group or a fused ring formed by the combination thereof.

Also, Formula 1 is represented by any one of Formulas 2 to 4

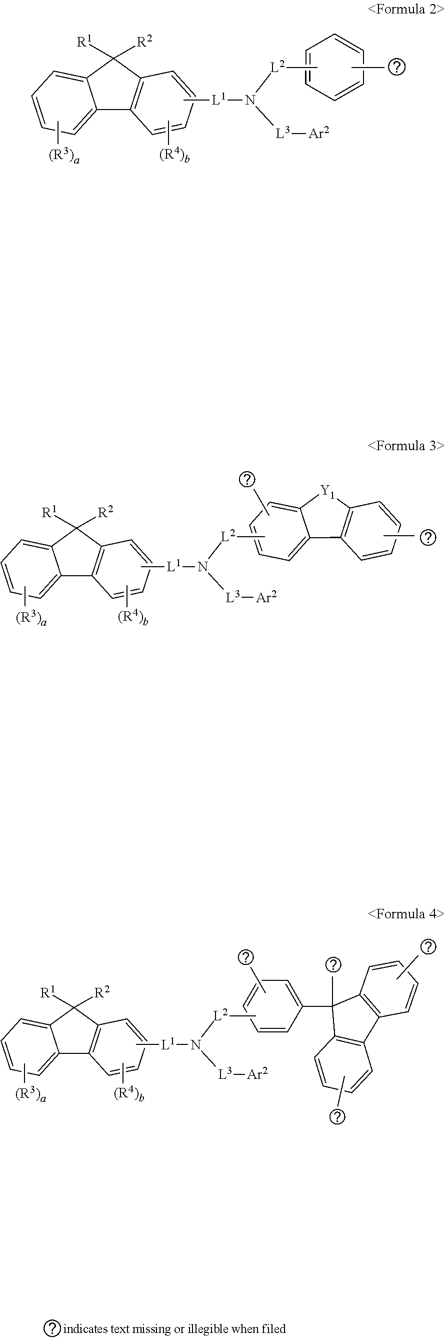

Wherein:

1) R¹, R², R³, R⁴, L¹, L², L³, Ar², a and b are the same as defined in Formula 1,

2) R⁵, R⁶, R⁷, R⁸ and R⁹ are the same as the definition of R³ in Formula 1,

3) c is an integer of 0 to 5, d is an integer of 0 to 3, e, f and g are independently an integer of 0 to 4,

4) R^{a} is selected from the group consisting of a C₁-C₅₀ alkyl group; a C₆-C₆₀ aryl group; fluorenyl group; a C₂-C₆₀ heterocyclic group including at least one heteroatom of O, N, S, Si or F;

Wherein in case R^{a} is an aryl group, it may be preferably a C₆-C₃₀ aryl group, more preferably a C₆-C₂₅ aryl group, for example, phenylene, biphenyl, naphthalene, terphenyl, etc.

Wherein in case R^{a} is a heterocyclic group, it may be preferably a C₂˜C₃₀ heterocyclic group, more preferably a C₂˜C₂₄ heterocyclic group, for example, pyrazine, thiophene, pyridine, pyrimidoindole, 5-phenyl-5H-pyrimido[5,4-b]indole, quinazoline, benzoquinazoline, carbazole, dibenzoquinazoline, dibenzofuran, benzothienopyrimidine, benzofuropyrimidine, phenothiazine, phenylphenothiazine, etc.

5) Y₁ is O, S or CR′R″,

6) wherein R′ and R″ are each independently selected from the group consisting of a C₆-C₆₀ aryl group; fluorenyl group; a C₂-C₆₀ heterocyclic group including at least one heteroatom of O, N, S, Si or F; a fused ring group of a C₃-C₆₀ aliphatic ring and a C₆-C₆₀ aromatic ring; and -L′-N(Rₐ)(R_{b}); or R′ and R″ are bonded to each other to form a C₆-C₆₀ aromatic ring; fluorenyl group; a C₂-C₆₀ heterocyclic group including at least one heteroatom of O, N, S, Si or F; or a fused ring group of a C₃-C₆₀ aliphatic ring and a C₆-C₆₀ aromatic ring;

Wherein in case R′ and R″ are an aryl group, they may be preferably a C₆-C₃₀ aryl group, more preferably a C₆-C₂₅ aryl group, for example, phenylene, biphenyl, naphthalene, terphenyl, etc.

Wherein in case R′ and R″ are a heterocyclic group, they may be preferably a C₂˜C₃₀ heterocyclic group, more preferably a C₂˜C₂₄ heterocyclic group,

Wherein in case R′ and R″ are a fused ring groups, they may be preferably a fused ring group of a C₃-C₃₀ aliphatic ring and a C₆-C₃₀ aromatic ring, more preferably a fused ring group of a C₃-C₂₄ aliphatic ring and a C₆-C₂₄ aromatic ring.

7) wherein L′, Rₐ and R_{b} are the same as defined in Formula 1.

Also, Formula 1 is represented by any one of Formulas 5 to 9.

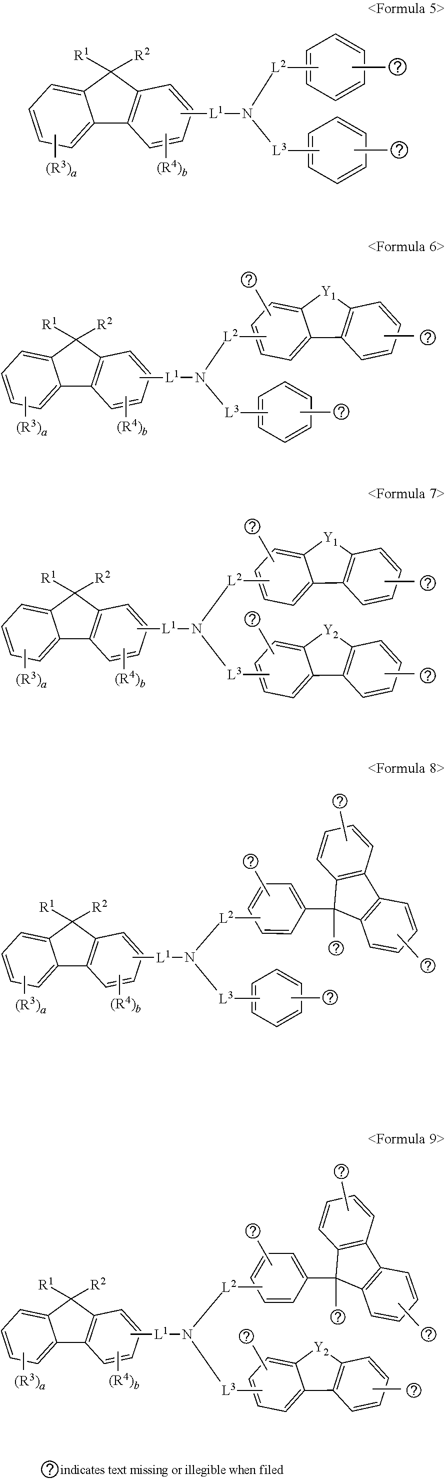

Wherein:

1) R¹, R², R³, R⁴, L¹, L², L³, a and b are the same as defined in Formula 1,

2) R⁵, R⁶, R⁷, R⁸, R⁹, c, d, e, f, g and Y₁ are the same as defined in Formula 2 to 4,

3) R¹′, R²′ and R³′ are the same as the definition of R³ in Formula 1,

4) a′ is an integer of 0 to 5, b′ is an integer of 0 to 3, c′ is an integer of 0 to 4,

5) Y₂ is O or S.

Also, Formula 1 is represented by any one of the following compounds P-1 to P-42.

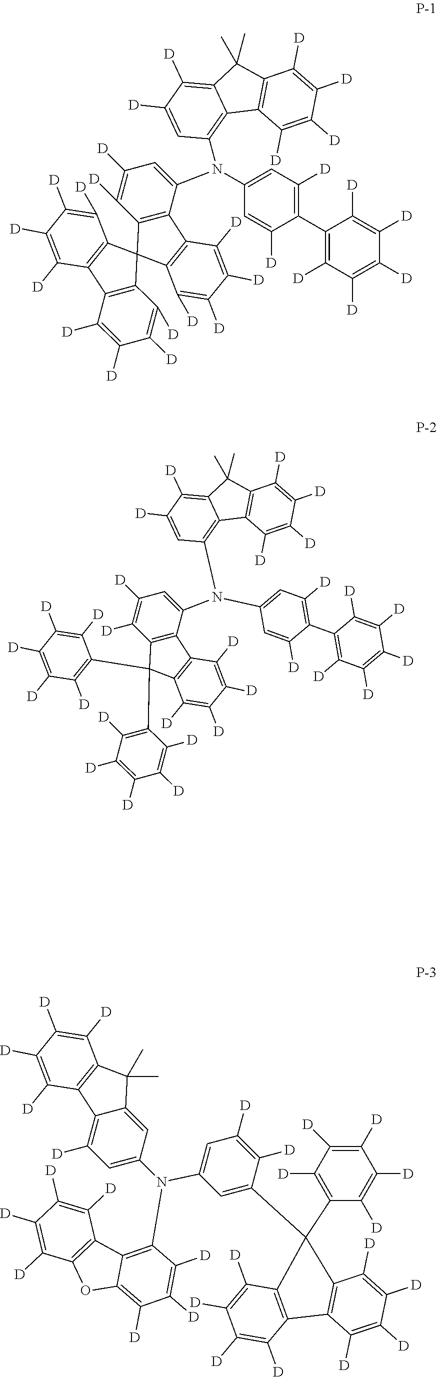

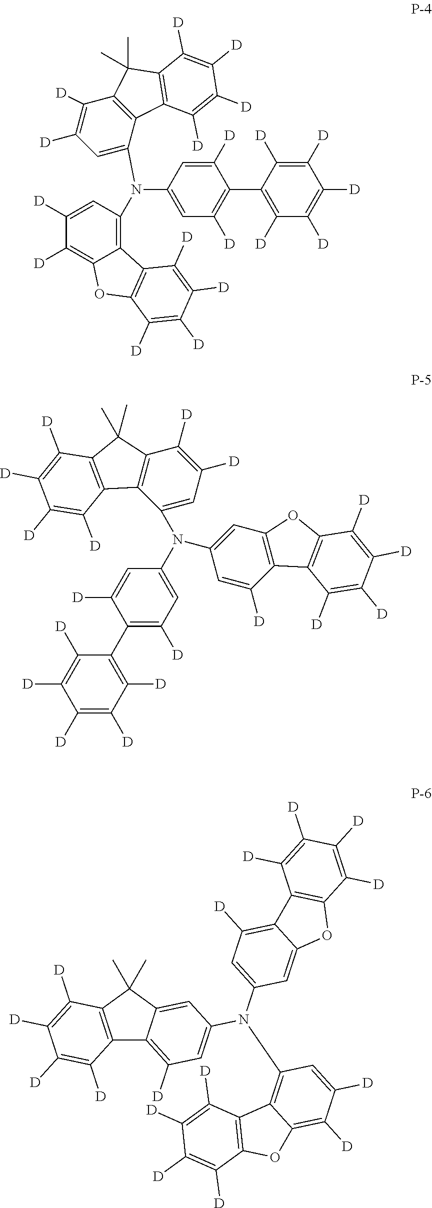

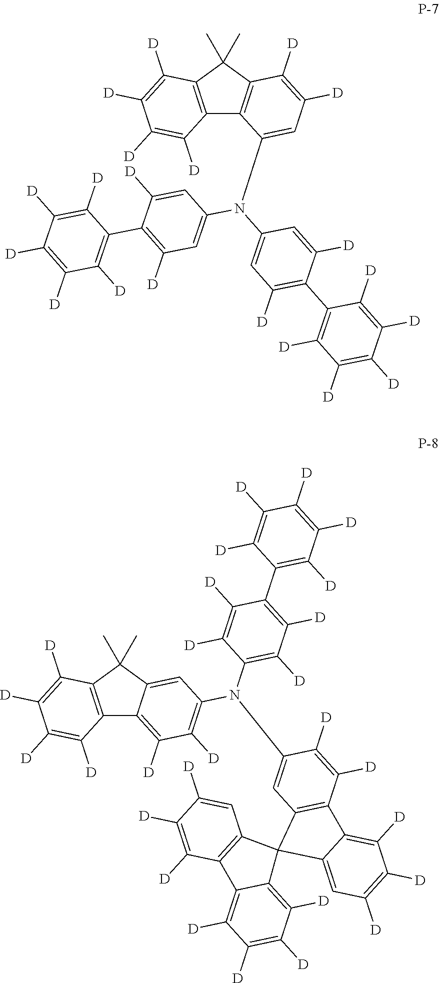 [SMILES: [2H]c1cc(N(c2cc([2H])c(-c3c([2H])c([2H])c([2H])c([2H])c3[2H])c([2H])c2)c2cc([2H])c(-c3c([2H])c([2H])c([2H])c([2H])c3[2H])c([2H])c2)c2c(c1[2H])C(C)(C)c1c([2H])c([2H])c([2H])c([2H])c1-2 ; [2H]c1cc2c(c([2H])c1[2H])-c1c(cc(N(c3cc4c(c([2H])c3[2H])-c3c([2H])c([2H])c([2H])c([2H])c3C4(C)C)c3c([2H])c([2H])c(-c4c([2H])c([2H])c([2H])c([2H])c4[2H])c([2H])c3[2H])c([2H])c1[2H])C21c2cc([2H])c([2H])c([2H])c2-c2c([2H])c([2H])c([2H])c([2H])c21]

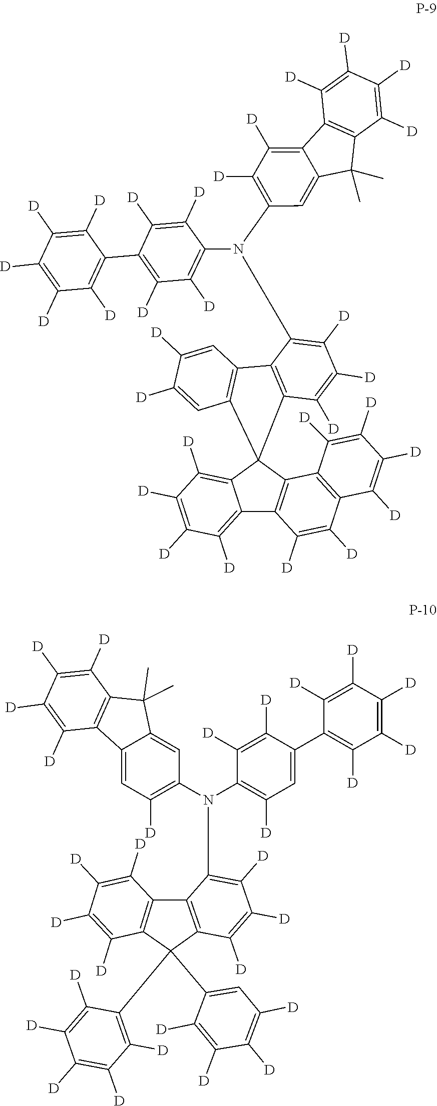

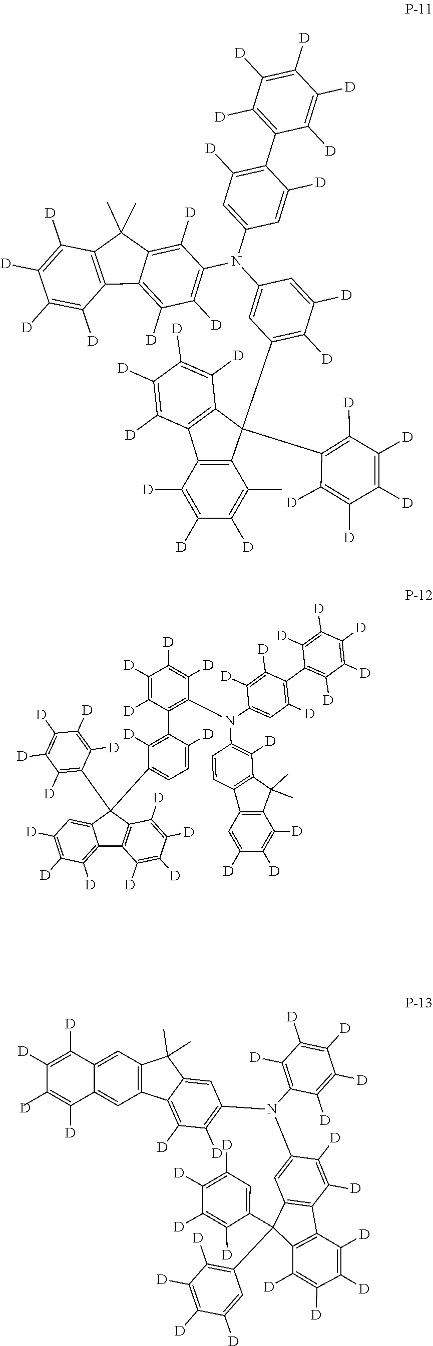

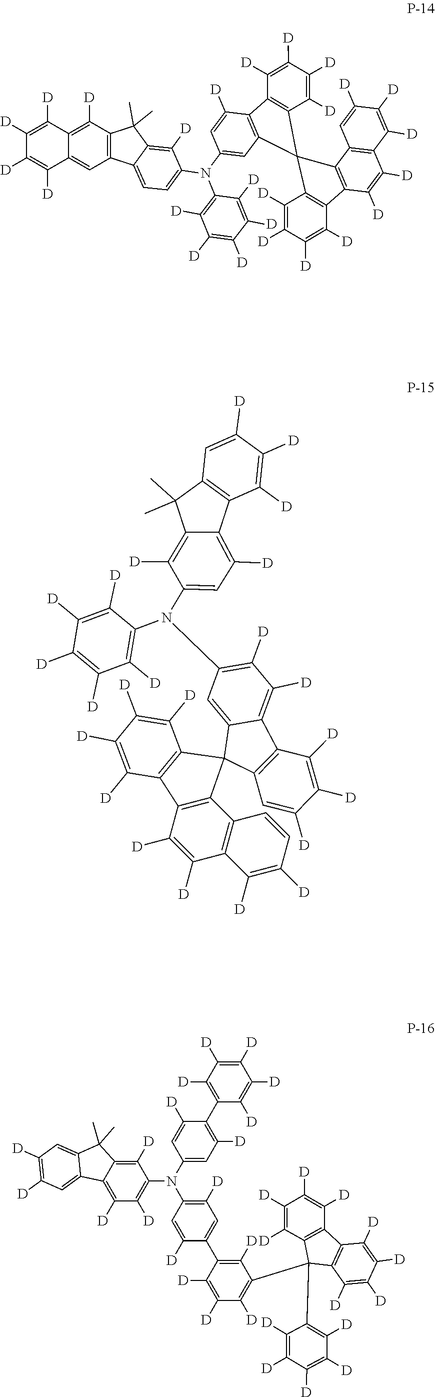

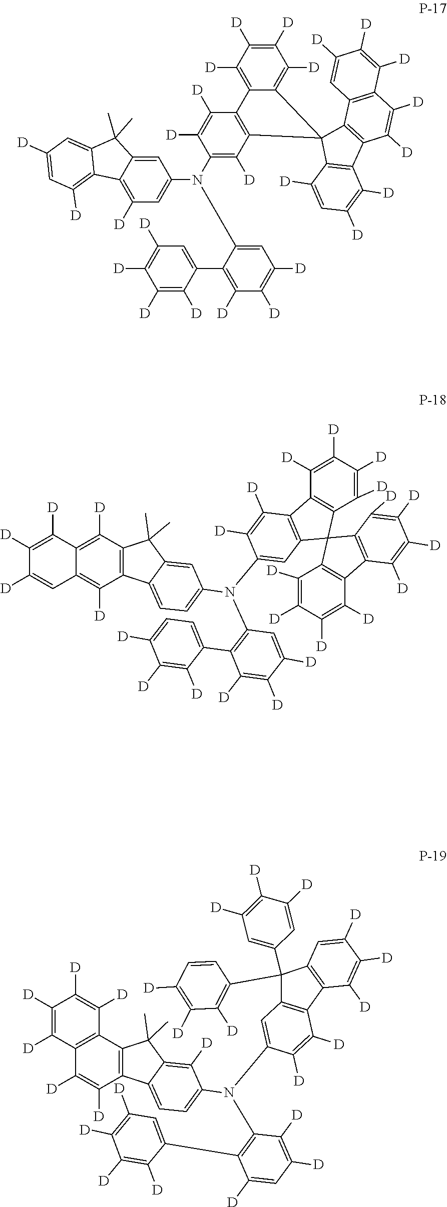

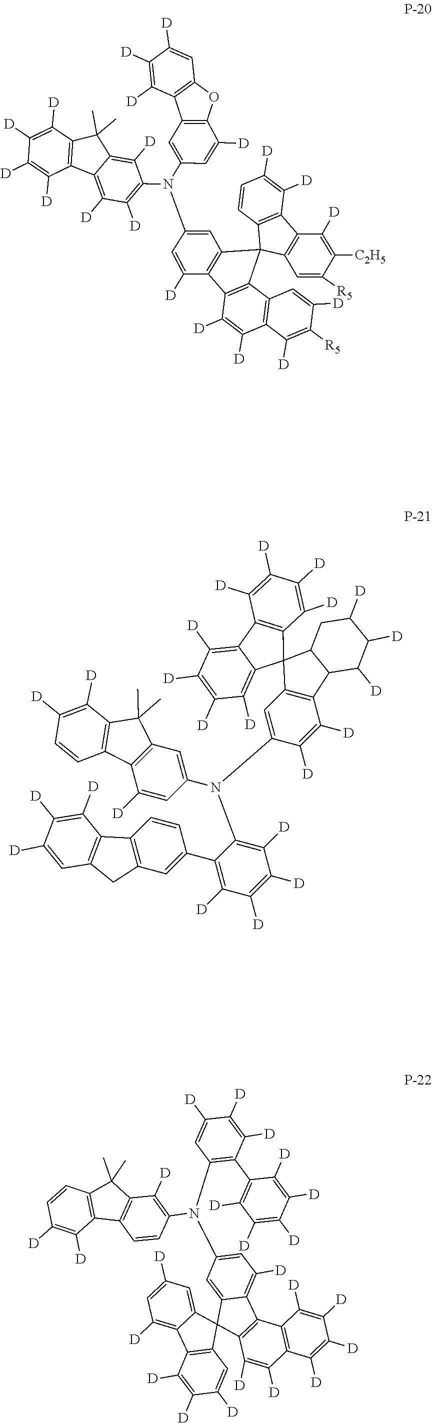

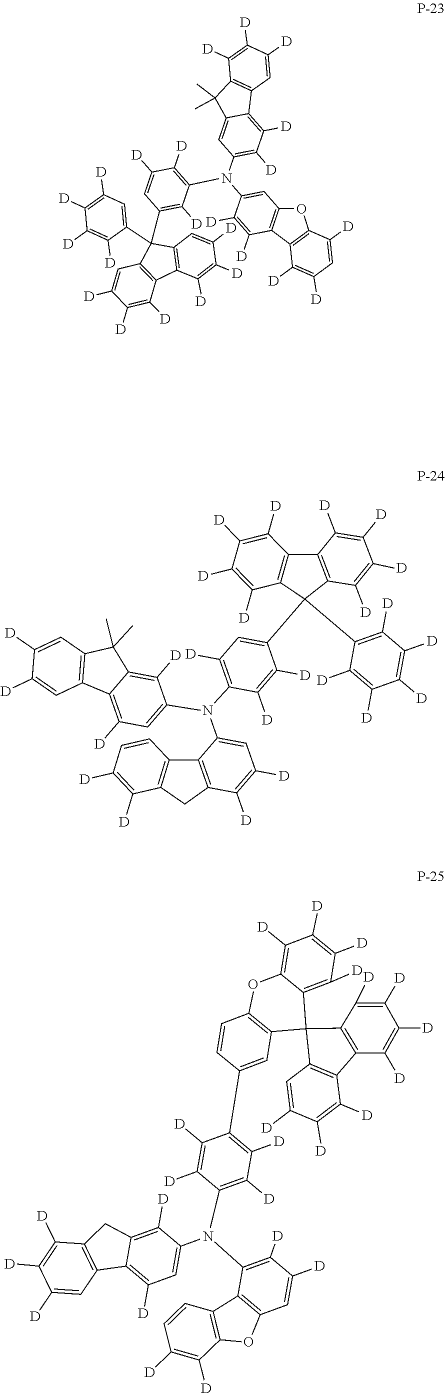

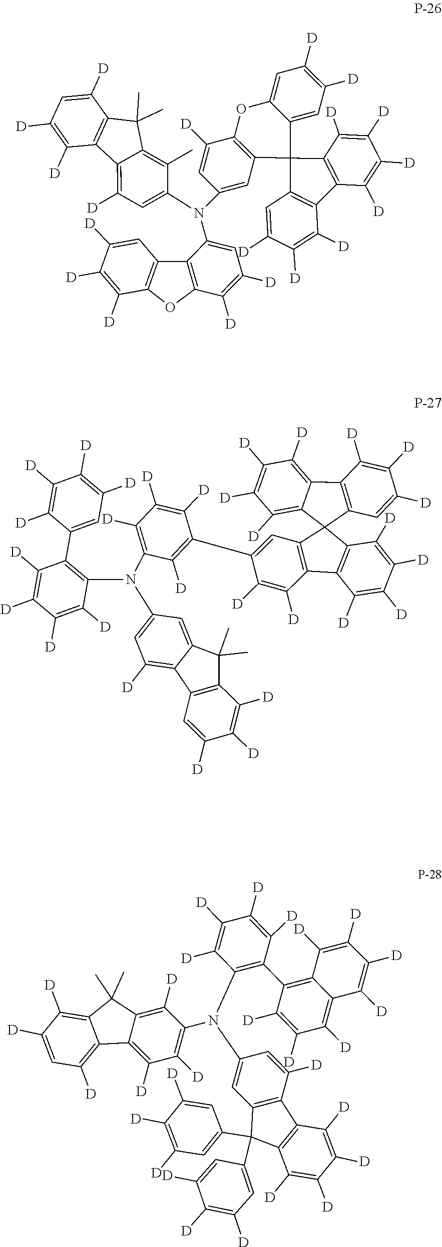

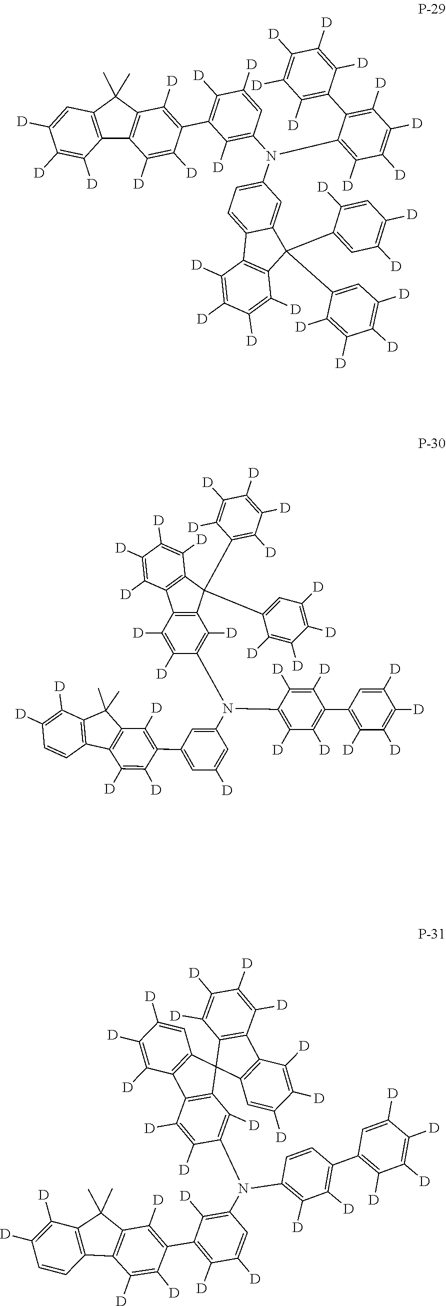

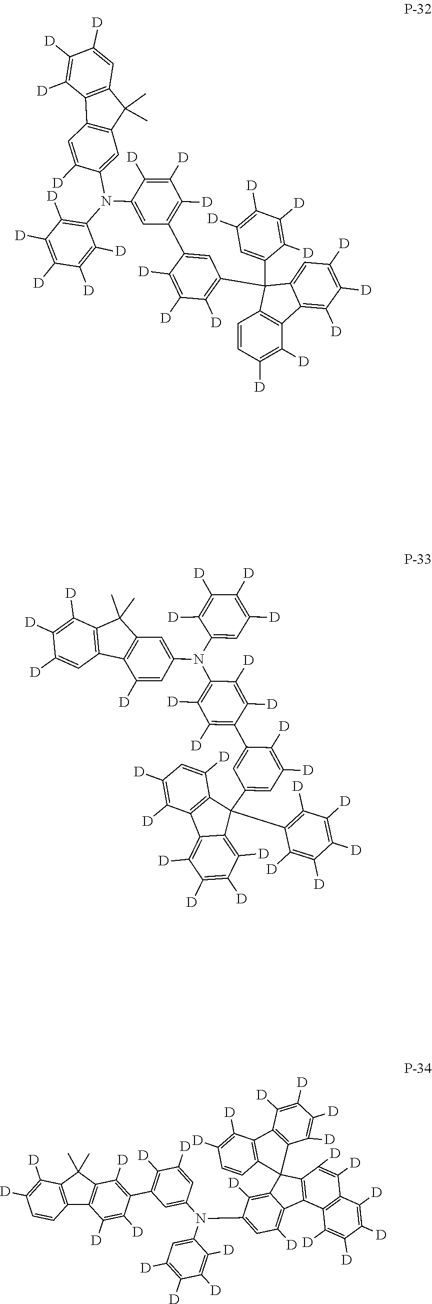

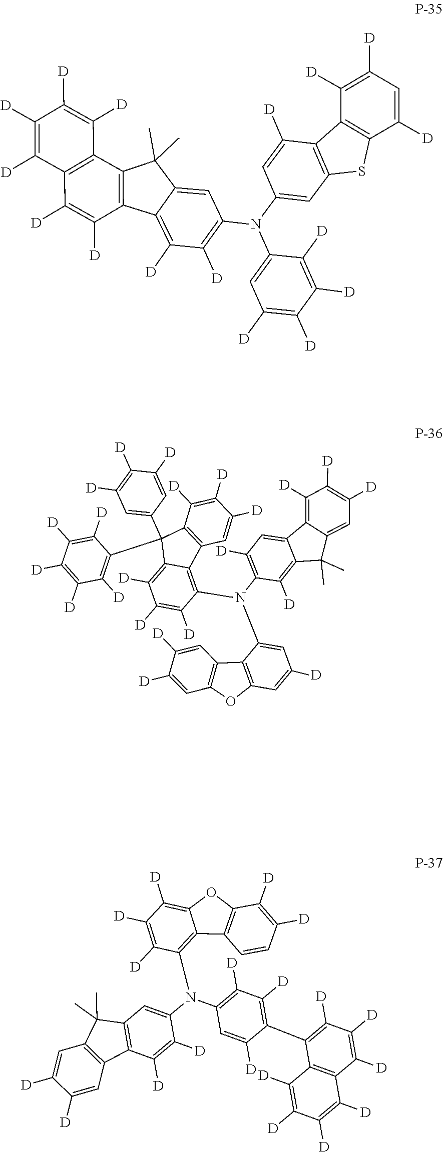

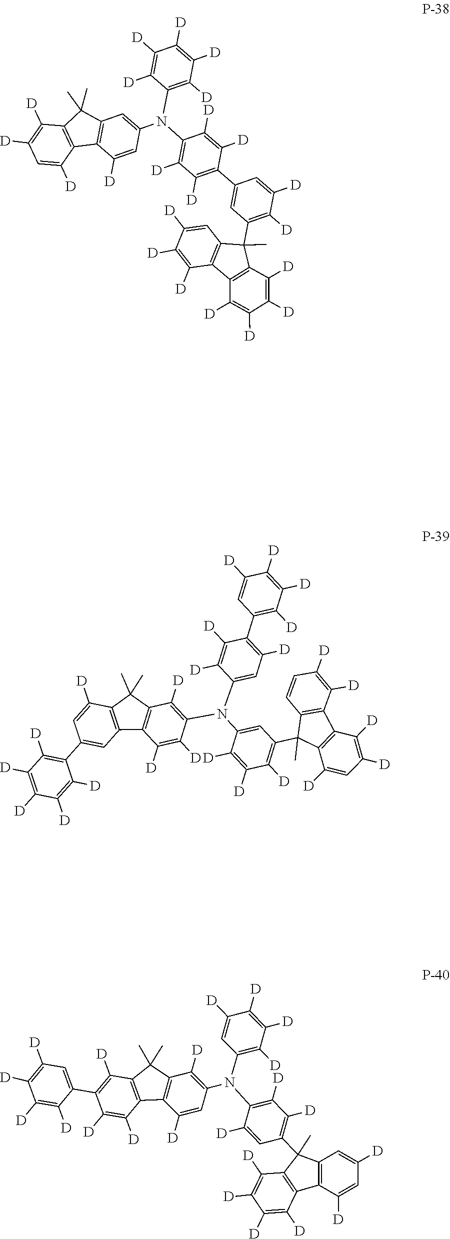

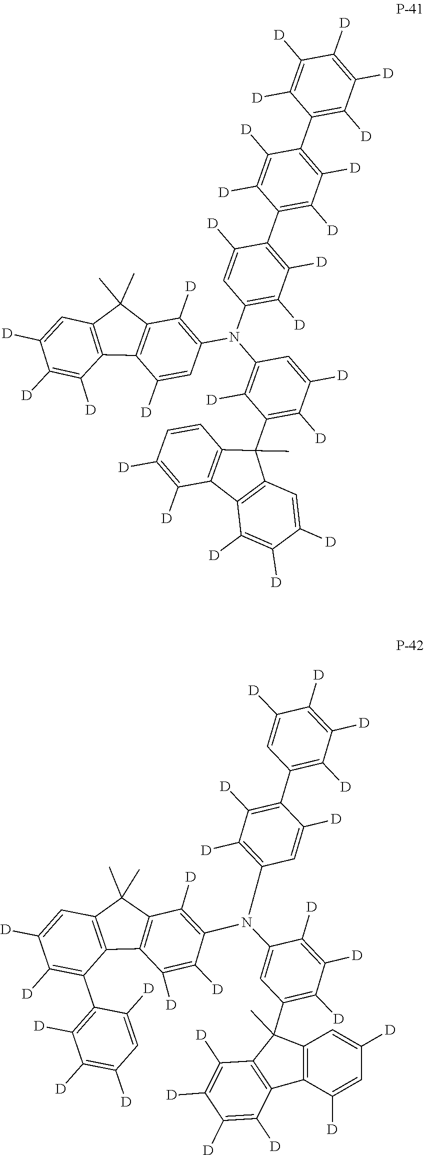 [SMILES: [2H]c1cc2c(c([2H])c1[2H])-c1c([2H])cc(N(c3cc([2H])c(-c4c([2H])c([2H])c(-c5c([2H])c([2H])c([2H])c([2H])c5[2H])c([2H])c4[2H])c([2H])c3[2H])c3cc([2H])c([2H])c(C4(C)c5ccc([2H])c([2H])c5-c5c4cc([2H])c([2H])c5[2H])c3[2H])c([2H])c1C2(C)C ; [2H]c1cc([2H])c2c(c1)C(C)(c1cc(N(c3cc([2H])c(-c4cc([2H])c([2H])c([2H])c4[2H])c([2H])c3[2H])c3c([2H])c([2H])c4c(c3[2H])C(C)(C)c3cc([2H])c([2H])c(-c5c([2H])cc([2H])c([2H])c5[2H])c3-4)c([2H])c([2H])c1[2H])c1c([2H])c([2H])c([2H])c([2H])c1-2]

Also, the present invention provides a method for preparing the 59%-73% deuterated compound represented by Formula 1 comprising:
(a) a step of forming a first reactant by dissolving the compound represented by Formula 1 in perdeuterated benzene (benzene-D₆);
(b) a step of forming a second reactant by adding deuterium-triflic acid (CF₃SO₃D) to the first reactant;
(c) a step deuterated by reacting the second reactant at 80° C. for 3 hours to 18 hours;
(d) a step quenched by adding Na₂CO₃ in D₂O, after the reaction is completed, the second reactant is cooled to room temperature,
(e) a step to obtain the deuterated compound represented by Formula 1 after concentrating the organic solvent of the second reactant, recrystallization with toluene and acetone solvent.

In step (c), the deuterated reaction time may be 3 hours to 18 hours, and preferably performed for 3 hours.

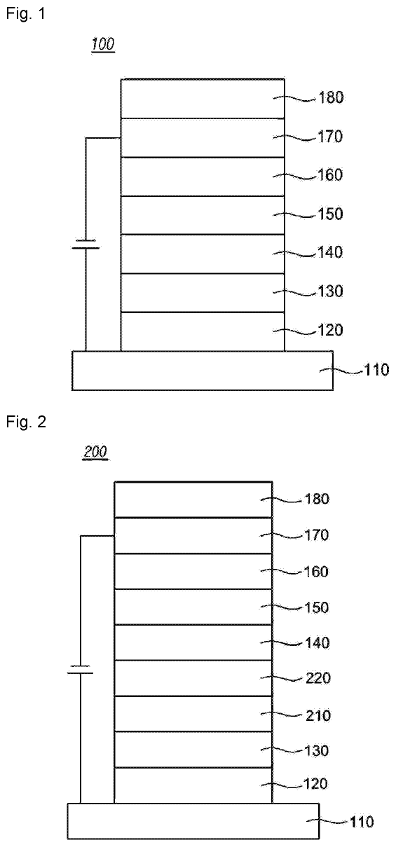

Referring to FIG. 1, the organic electric element (100) according to the present invention includes a first electrode (110), a second electrode (170), and an organic material layer including a single compound or 2 or more compounds represented by Formula A between the first electrode (110) and the second electrode (170). In this case, the first electrode (110) may be an anode, and the second electrode (170) may be a cathode. In the case of an inverted type, the first electrode may be a cathode and the second electrode may be an anode.

The organic material layer may sequentially include a hole injection layer (120), a hole transport layer (130), an emitting layer (140), an electron transport layer (150), and an electron injection layer (160) on the first electrode (110). In this case, the remaining layers except for the emitting layer (140) may not be formed. It may further include a hole blocking layer, an electron blocking layer, an emitting-auxiliary layer (220), a buffer layer (210), etc. and the electron transport layer (150) and the like may serve as a hole blocking layer. (See FIG. 2)

Also, the organic electric element according to an embodiment of the present invention may further include a protective layer or a light efficiency enhancing layer (180). The light efficiency enhancing layer may be formed on one of both surfaces of the first electrode not in contact with the organic material layer or on one of both surfaces of the second electrode not in contact with the organic material layer.

The compound according to an embodiment of the present invention applied to the organic material layer may be used as the hole injection layer (120), the hole transport layer (130), the emitting-auxiliary layer (220), electron transport auxiliary layer, the electron transport layer (150), and an electron injection layer (160), a host or dopant of the emitting layer (140) or a material for the light efficiency enhancing layer. Preferably, for example, the compound according to Formula A of the present invention may be used as a material for an emitting auxiliary layer or a hole transport layer.

The organic material layer may include 2 or more stacks including a hole transport layer, an emitting layer and an electron transport layer sequentially formed on the anode, further include a charge generation layer formed between the 2 or more stacks (see FIG. 3).

Otherwise, even with the same core, the band gap, electrical characteristics, interface characteristics, etc. may vary depending on which position the substituent is bonded to, therefore the choice of core and the combination of sub-substituents bound thereto are also very important, and in particular, when the optimal combination of energy levels and T1 values and unique properties of materials(mobility, interfacial characteristics, etc.) of each organic material layer is achieved, a long lifespan and high efficiency can be achieved at the same time.

The organic electroluminescent device according to an embodiment of the present invention may be manufactured using a PVD (physical vapor deposition) method. For example, depositing a metal or a metal oxide having conductivity or an alloy thereof on a substrate to form an anode, and after forming an organic material layer including the hole injection layer (120), the hole transport layer (130), the emitting layer (140), the electron transport layer (150) and the electron injection layer (160) thereon, it can be prepared by depositing a material that can be used as a cathode thereon.

Also, in the present invention, the organic material layer is formed by any one of a spin coating process, a nozzle printing process, an inkjet printing process, a slot coating process, a dip coating process, and a roll-to-roll process, and the organic material layer provides an organic electric element comprising the compound as an electron transport material.

As another specific example, the compound of the same or different types of the compound represented by Formula 1 is mixed and used in the organic material layer.

Also, the present invention provides an emitting auxiliary layer composition comprising the compound represented by Formula A, and provides an organic electric element including the emitting auxiliary layer.

Also, the present invention provides a hole transport layer composition comprising the compound represented by Formula (A), and provides an organic electric element including the hole transport layer.

Also, the present invention provides an electronic device comprising a display device including the organic electric element; and a control unit for driving the display device;

In another aspect, the organic electric element is at least one of an organic electroluminescent device, an organic solar cell, an organic photo conductor, an organic transistor, and a device for monochromatic or white lighting. At this time, the electronic device may be a current or future wired/wireless communication terminal, and covers all kinds of electronic devices including mobile communication terminals such as mobile phones, a personal digital assistant(PDA), an electronic dictionary, a point-to-multipoint(PMP), a remote controller, a navigation unit, a game player, various kinds of TVs, and various kinds of computers.

Hereinafter, a synthesis example of the compound represented by Formula A of the present invention and a manufacturing example of an organic electric element of the present invention will be described in detail with reference to Examples, but the present invention is not limited to the following Examples.

### Synthesis Example 1

The compound (final product 1-1) represented by Formula 1 according to the present invention is prepared by reacting Sub 1 with Sub 2 as shown in Reaction Scheme 1.

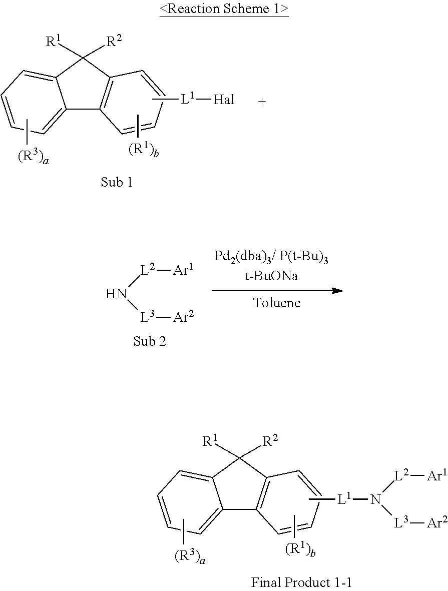

The deuterated compound of compound (final product 1-1) represented by Formula 1 according to the present invention is prepared by dissolving in perdeuterated benzene (benzene-D₆), adding deuterium-triflic acid (CF₃SO₃D), and reacting at a temperature of 80° C. for 3 hours to 18 hours, and more preferably 3 hours.

### 1. Synthesis Example of P-2

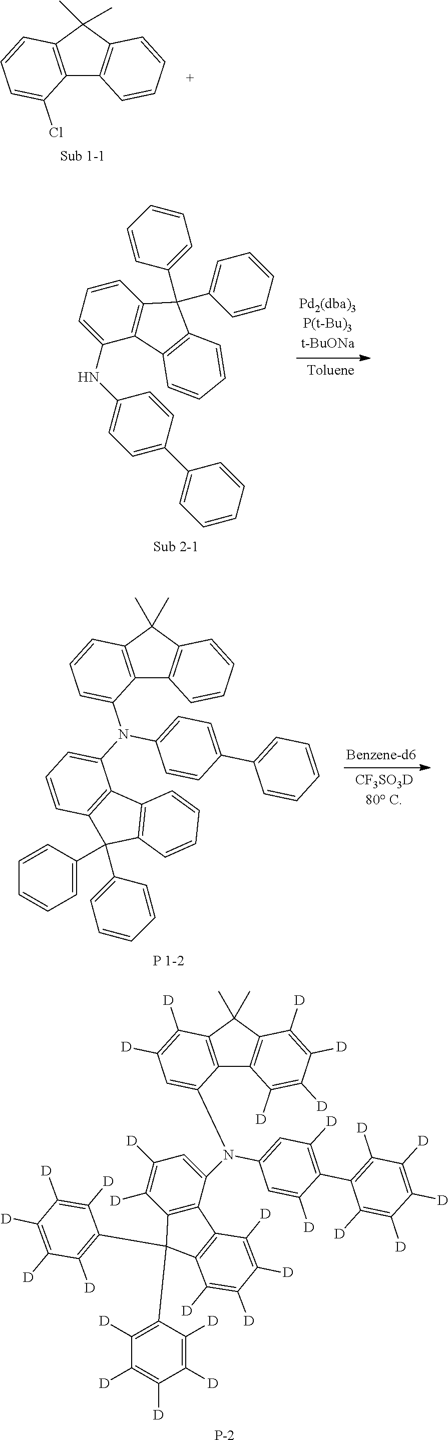

(1) Synthesis of P 1-2

Sub 1-1 (10.5 g, 45.9 mmol), Sub 2-1 (22.3 g, 45.9 mmol), Pd2(dba)3 (1.3 g, 1.4 mmol), t-BuONa (8.8 g, 91.8 mmol) were placed in a round-bottom flask and dissolved in anhydrous Toluene (210 mL), then P(t-Bu)₃ (50 wt % Sol.) (1.11 mL, 2.8 mmol) was added and heated and stirred at 110° C. for about 4 hours. After confirming the completion of the reaction by TLC, cooled to room temperature and extracted with CH₂Cl₂ and water. The separated organic layer was dried over MgSO₄, concentrated, and the resulting compound was recrystallized using silicagel column (Hexane: CH₂Cl₂=4:1) to obtain 25.8 g (yield: 83%) of P 1-2.

(2) Synthesis of P-2

P 1-2 (15.0 g, 22.1 mmol) obtained in the above synthesis was dissolved in perdeuterated benzene (C₆D₆) (167.6 g, 1,991.5 mmol) and CF₃SO₃D (16.6 g, 110.6 mmol) was added thereto, followed by a temperature of 80° C. reacted for 3 hours to form a deuterated material. Periodically take a sample, measure the degree of deuterium by LC-MS, and after the deuterium exchange reaction is completed at a desired substitution rate, cool to room temperature, quench by adding Na₂CO₃ in D₂O, and concentrate the organic solvent. Recrystallization using toluene and acetone solvent gave 14.7 g (yield: 94%) of deuterated compound P-2. The final mass was determined by LC-MS to confirm to be 72.9% deuterated.

### 2. Synthesis Example of P-3

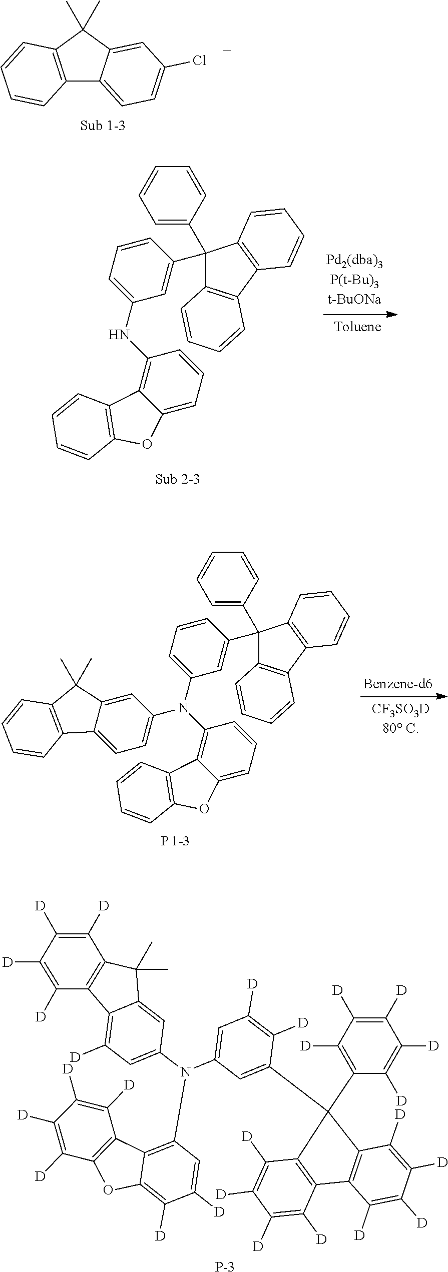

(1) Synthesis of P 1-3

Sub 1-3 (11.3 g, 49.4 mmol), Sub 2-3 (24.7 g, 49.4 mmol), Pd₂(dba)₃ (1.4 g, 1.5 mmol), t-BuONa (9.5 g, 98.8 mmol) were placed in a round-bottom flask and dissolved in anhydrous Toluene (230 mL), then P(t-Bu)₃ (50 wt % Sol.) (1.2 mL, 2.96 mmol) was added and heated and stirred at 110° C. for about 4 hours. After confirming the completion of the reaction by TLC, cooled to room temperature and extracted with CH₂Cl₂ and water. The separated organic layer was dried over MgSO₄, concentrated, and the resulting compound was recrystallized using silicagel column (Hexane: CH₂Cl₂=4:1) to obtain 29.0 g (yield: 85%) of P 1-3.

(2) Synthesis of P-3

P 1-3 (18.5 g, 26.7 mmol) obtained in the above synthesis was dissolved in perdeuterated benzene (C₆D₆) (202.5 g, 2406.5 mmol) and CF₃SO₃D (20.1 g, 133.7 mmol) was added thereto, followed by a temperature of 80° C. reacted for 3 hours to form a deuterated material. Periodically take a sample, measure the degree of deuterium by LC-MS, and after the deuterium exchange reaction is completed at a desired substitution rate, cool to room temperature, quench by adding Na₂CO₃ in D₂O, and concentrate the organic solvent. Recrystallization using toluene and acetone solvent gave 18.4 g (yield: 96%) of deuterated compound P-3. The final mass was determined by LC-MS to confirm to be 70.2% deuterated.

### 3. Synthesis Example of P-6

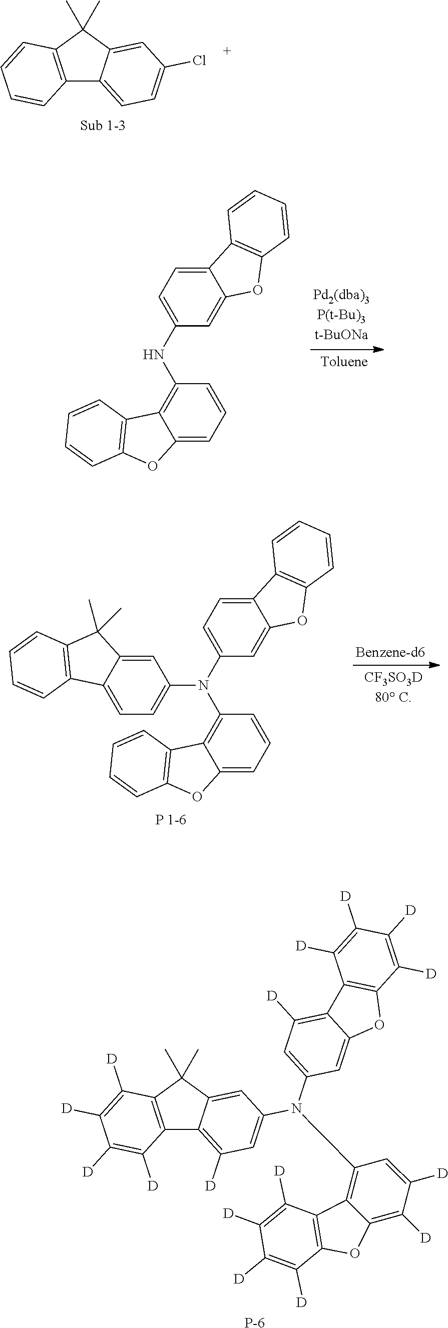

(1) Synthesis of P 1-6

Sub 1-3 (9.9 g, 43.3 mmol), Sub 2-6 (15.1 g, 43.3 mmol), Pd₂(dba)₃ (1.2 g, 1.3 mmol), t-BuONa (8.3 g, 86.6 mmol) were placed in a round-bottom flask and dissolved in anhydrous Toluene (200 mL), then P(t-Bu)₃ (50 wt % Sol.) (1.05 mL, 0.5 mmol) was added and heated and stirred at 110° C. for about 4 hours. After confirming the completion of the reaction by TLC, cooled to room temperature and extracted with CH₂Cl₂ and water. The separated organic layer was dried over MgSO₄, concentrated, and the resulting compound was recrystallized using silicagel column (Hexane: CH₂Cl₂=4:1) to obtain 19.2 g (yield: 82%) of P 1-6.

(2) Synthesis of P-6

P 1-6 (19.2 g, 35.5 mmol) obtained in the above synthesis was dissolved in perdeuterated benzene (C₆D₆) (268.5 g, 3190.3 mmol) and CF₃SO₃D (26.6 g, 177.2 mmol) was added thereto, followed by a temperature of 80° C. reacted for 3 hours to form a deuterated material. Periodically take a sample, measure the degree of deuterium by LC-MS, and after the deuterium exchange reaction is completed at a desired substitution rate, cool to room temperature, quench by adding Na₂CO₃ in D₂O, and concentrate the organic solvent. Recrystallization using toluene and acetone solvent gave 18.4 g (yield: 93%) of deuterated compound P-6. The final mass was determined by LC-MS to confirm to be 59.2% deuterated.

### 4. Synthesis Example of P-7

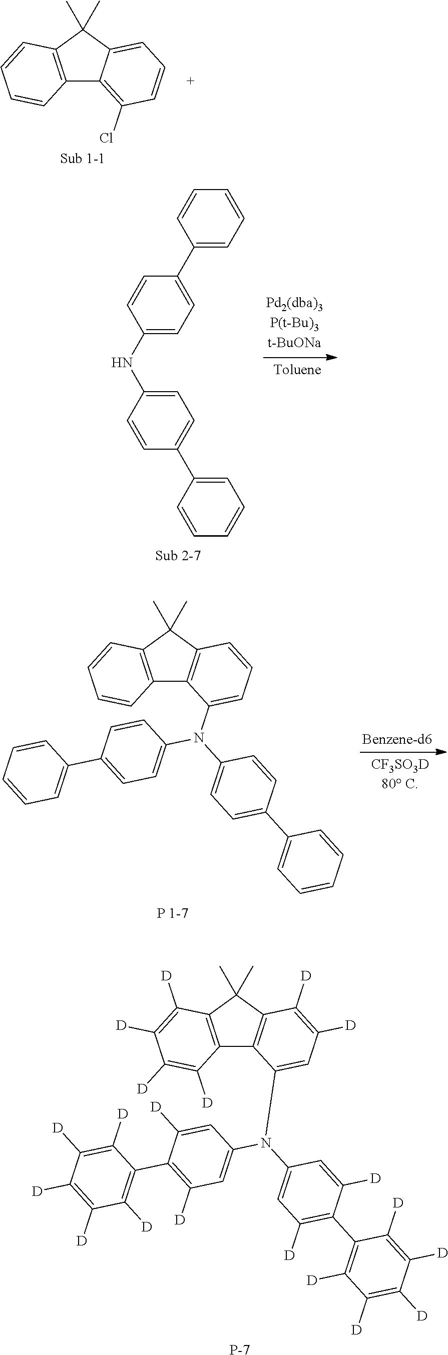

(1) Synthesis of P 1-7

Sub 1-1 (10.2 g, 44.6 mmol), Sub 2-7 (14.3 g, 44.6 mmol), Pd₂(dba)₃ (1.2 g, 1.3 mmol), t-BuONa (8.6 g, 89.2 mmol) were placed in a round-bottom flask and dissolved in anhydrous Toluene (200 mL), then P(t-Bu)₃ (50 wt % Sol.) (1.1 mL, 2.7 mmol) was added and heated and stirred at 110° C. for about 4 hours. After confirming the completion of the reaction by TLC, cooled to room temperature and extracted with CH₂Cl₂ and water. The separated organic layer was dried over MgSO₄, concentrated, and the resulting compound was recrystallized using silicagel column (Hexane: CH₂Cl₂=4:1) to obtain 20.6 g (yield: 90%) of P 1-7.

(2) Synthesis of P-7

P 1-7 (15.0 g, 29.2 mmol) obtained in the above synthesis was dissolved in perdeuterated benzene (C₆D₆) (221.2 g, 2,628.1 mmol) and CF₃SO₃D (21.9 g, 146 mmol) was added thereto, followed by a temperature of 80° C. reacted for 3 hours to form a deuterated material. Periodically take a sample, measure the degree of deuterium by LC-MS, and after the deuterium exchange reaction is completed at a desired substitution rate, cool to room temperature, quench by adding Na₂CO₃ in D₂O, and concentrate the organic solvent. Recrystallization using toluene and acetone solvent gave 14.8 g (yield: 95%) of deuterated compound P-7. The final mass was determined by LC-MS to confirm to be 64.5% deuterated.

### Comparative Synthesis Example 1

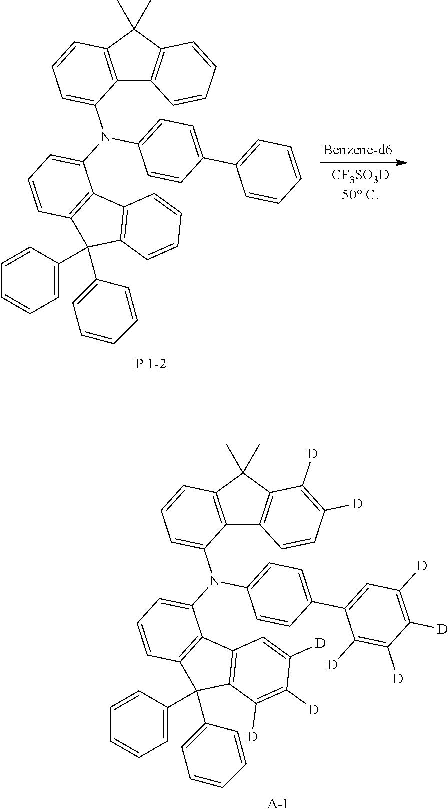 [SMILES: CC1(C)c2ccccc2-c2c(N(c3ccc(-c4ccccc4)cc3)c3cccc4c3-c3ccccc3C4(c3ccccc3)c3ccccc3)cccc21 ; [2H]c1cc(-c2ccc(N(c3cccc4c3-c3ccc([2H])c([2H])c3C4(C)C)c3cccc4c3-c3cc([2H])c([2H])c([2H])c3C4(c3ccccc3)c3ccccc3)cc2)c([2H])c([2H])c1[2H] ; "A-1"]

1. Synthesis of Comparative Compound A-1

P 1-2 (10.8 g, 15.9 mmol) obtained in the above synthesis was dissolved in perdeuterated benzene (C₆D₆) (120.7 g, 1,433.9 mmol) and CF₃SO₃D (12.0 g, 79.7 mmol) was added thereto, followed by a temperature of 50° C. reacted for 20 hours to form a deuterated material. After the reaction is completed at a desired substitution rate, cool to room temperature, quench by adding Na₂CO₃ in D₂O, and concentrate the organic solvent. Recrystallization using toluene and acetone solvent gave 10.0 g (yield: 92%) of deuterated compound A-1. The final mass was determined by LC-MS to confirm to be 23.1% deuterated.

As can be seen from the results of Comparative Synthesis Example 1, it can be seen that the conventionally known general deuterium substitution method has a long reaction time and is difficult to control the substitution rate with a significantly lower deuterium substitution rate compared to the manufacturing method of the present invention. In the manufacturing method described in the present invention, the reaction time is shortened by reacting at a high temperature compared to the existing reaction temperature for 3 to 18 hours, more preferably 3 hours, and a deuterated compound having an improved substitution rate can be obtained.

Meanwhile, FD-MS values of compounds P-1 to P-42 of the present invention prepared according to the above synthesis examples are shown in Table 1.

TABLE 1

| compound | FD-MS | compound | FD-MS |
|---|---|---|---|
| P-1 | m/z = 702.46(C₅₂H₁₀D₂₇N = 703.04) | P-2 | m/z = 706.49(C₅₂H₁₀D₂₉N = 707.07) |
| P-3 | m/z = 717.45(C₅₂H₁₁D₂₆NO = 718.03) | P-4 | m/z = 546.34(C₃₉H₁₀D₁₉NO = 546.78) |
| P-5 | m/z = 545.34(C₃₉H₁₁D₁₈NO = 545.78) | P-6 | m/z = 557.30(C₃₉H₁₁D₁₆NO₂ = 557.75) |
| P-7 | m/z = 533.37(C₃₉H₁₁D₂₀N = 533.81) | P-8 | m/z = 702.46(C₅₂H₁₀D₂₇N = 703.04) |
| P-9 | m/z = 755.50(C₅₆H₉D₃₀N = 756.12) | P-10 | m/z = 706.49(C₅₂H₁₀D₂₉N = 707.07) |
| P-11 | m/z = 719.50(C₅₃H₁₃D₂₈N = 720.09) | P-12 | m/z = 783.53(C₅₈H₁₃D₃₀N = 784.17) |
| P-13 | m/z = 676.45(C₅₀H₁₂D₂₅N = 677.01) | P-14 | m/z = 724.45(C₅₄H₁₂D₂₅N = 725.05) |
| P-15 | m/z = 672.42(C₅₀H₁₂D₂₃N = 672.98) | P-16 | m/z = 784.53(C₅₈H₁₂D₃₁N = 785.18) |
| P-17 | m/z = 751.47(C₅₆H₁₃D₂₆N = 752.09) | P-18 | m/z = 750.47(C₅₆H₁₄D₂₅N = 751.09) |
| P-19 | m/z = 752.48(C₅₆H₁₆D₂₅N = 753.10) | P-20 | m/z = 761.43(C₅₆H₁₅D₂₂NO = 762.05) |
| P-21 | m/z = 789.45(C₅₈H₁₅D₂₄NO = 790.10) | P-22 | m/z = 748.45(C₅₆H₁₆D₂₃N = 749.08) |
| P-23 | m/z = 714.43(C₅₂H₁₄D₂₃NO = 715.01) | P-24 | m/z = 715.44(C₅₂H₁₃D₂₄NO = 716.02) |
| P-25 | m/z = 806.45(C₅₈H₁₄D₂₅NO₂ = 807.11) | P-26 | m/z = 726.40(C₅₂H₁₄D₂₁NO₂ = 726.99) |
| P-27 | m/z = 780.51(C₅₈H₁₂D₂₉N = 781.15) | P-28 | m/z = 755.5(C₅₆H₁₃D₂₈N = 756.12) |
| P-29 | m/z = 784.53(C₅₈H₁₂D₃₁N = 785.18) | P-30 | m/z = 783.53(C₅₈H₁₃D₃₀N = 784.17) |
| P-31 | m/z = 780.51(C₅₈H₁₂D₂₉N = 781.15) | P-32 | m/z = 701.46(C₅₂H₁₅D₂₄N = 702.04) |
| P-33 | m/z = 703.47(C₅₂H₁₃D₂₆N = 704.05) | P-34 | m/z = 750.47(C₅₆H₁₄D₂₅N = 751.09) |
| P-35 | m/z = 533.29(C₃₇H₁₁D₁₆NS = 533.79) | P-36 | m/z = 713.43(C₅₂H₁₅D₂₂NO = 714.01) |
| P-37 | m/z = 596.36(C₄₃H₁₂D₁₉NO = 596.84) | P-38 | m/z = 637.43(C₄₇H₁₅D₂₂N = 637.95) |
| P-39 | m/z = 716.48(C₅₃H₁₆D₂₅N = 717.07) | P-40 | m/z = 637.43(C₄₇H₁₅D₂₂N = 637.95) |
| P-41 | m/z = 716.48(C₅₃H₁₆D₂₅N = 717.07) | P-42 | m/z = 716.48(C₅₃H₁₆D₂₅N = 717.07) |

Manufacturing Evaluation of Organic Electronic Elements

### Example 1

### Blue Organic Light Emitting Device (Emitting Auxiliary Layer)

After vacuum deposition of 2-TNATA to a thickness of 60 nm on the ITO layer (anode) formed on the glass substrate to form a hole injection layer, NPB was vacuum deposited on the hole injection layer to a thickness of 60 nm to form a hole transport layer. Then, the compound P-1 of the present invention was vacuum-deposited to a thickness of 20 nm on the hole transport layer to form an emitting auxiliary layer, and 9,10-di(naphthalen-2-yl)anthracene as a host and BD-052X (manufactured by Idemitsu Kosan) as a dopant were used in a weight ratio of 96:4 on the emitting auxiliary layer to form an emitting layer with a thickness of 30 nm. Subsequently, (1,1′-biphenyl-4-olato)bis(2-methyl-8-quinolinolato)aluminum (hereinafter, BAlq) was vacuum-deposited to a thickness of 10 nm to form a hole blocking layer, and on the hole blocking layer, bis(10-hydroxybenzo[h]quinolinato)beryllium (hereinafter, BeBq₂) was vacuum-deposited to a thickness of 40 nm to form an electron transport layer. Thereafter, LiF, which is an alkali metal halide, was deposited to a thickness of 0.2 nm to form an electron injection layer, and then Al was deposited to a thickness of 150 nm to form a cathode, thereby manufacturing an organic electroluminescent device.

### Example 2 to Example 20

### Blue Organic Electroluminescent Device (Emitting Auxiliary Layer)

An organic electroluminescent device was manufactured in the same manner as in Example 1, except that the compound of the present invention described in Table 2 was used instead of the compound P-1 of the present invention as an emitting auxiliary layer material.

### Comparative Example 1 and Comparative Example 2

An organic electroluminescent device was manufactured in the same manner as in Example 1, except that Comparative Compound 1 or Comparative Compound 2 described in Table 2 was used instead of Compound P-1 of the present invention as an emitting auxiliary layer material.

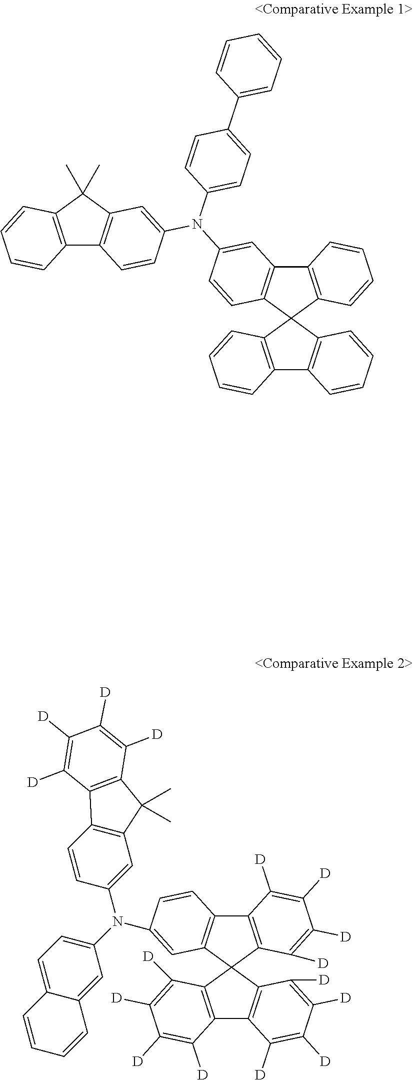 [SMILES: CC1(C)c2ccccc2-c2ccc(N(c3ccc(-c4ccccc4)cc3)c3ccc4c(c3)-c3ccccc3C43c4ccccc4-c4ccccc43)cc21 ; [2H]c1c([2H])c([2H])c2c(c1[2H])-c1ccc(N(c3ccc4c(c3)C3(c5c([2H])c([2H])c([2H])c([2H])c5-4)c4c([2H])c([2H])c([2H])c([2H])c4-c4c([2H])c([2H])c([2H])c([2H])c43)c3ccc4ccccc4c3)cc1C2(C)C]

By applying a forward bias DC voltage to the organic electroluminescent devices manufactured by Examples 1 to 20, Comparative Examples 1 and Comparative Example 2 of the present invention, Electroluminescence (EL) characteristics were measured with PR-650 from Photoresearch, and the T95 lifetime was measured using a lifetime measuring device manufactured by McScience at 500 cd/m² standard luminance, and the measurement results are shown in Table 2.

TABLE 2

| | | | Current | | | | | |
|---|---|---|---|---|---|---|---|---|
| | | Voltage | Density | Brightness | Efficiency | Lifetime | CIE | |
| | compound | (V) | (mA/cm²) | (cd/m²) | (cd/A) | T(95) | x | y |
| comparative | comparative | 5.6 | 7.9 | 500.0 | 6.3 | 82.4 | 0.132 | 0.100 |
| example(1) | compound1 | | | | | | | |
| comparative | comparative | 5.5 | 7.8 | 500.0 | 6.4 | 95.6 | 0.133 | 1.100 |
| example(2) | compound2 | | | | | | | |
| example(1) | P-1 | 5.3 | 7.7 | 500.0 | 6.5 | 118.6 | 0.132 | 0.100 |
| example(2) | P-2 | 5.4 | 7.8 | 500.0 | 6.4 | 117.7 | 0.131 | 0.100 |
| example(3) | P-3 | 5.4 | 7.8 | 500.0 | 6.4 | 116.8 | 0.133 | 0.100 |
| example(4) | P-4 | 5.4 | 7.7 | 500.0 | 6.5 | 117.6 | 0.132 | 0.100 |
| example(5) | P-5 | 5.3 | 7.6 | 500.0 | 6.6 | 118.8 | 0.132 | 0.100 |
| example(6) | P-6 | 5.5 | 7.8 | 500.0 | 6.4 | 118.3 | 0.130 | 0.100 |
| example(7) | P-7 | 5.3 | 7.5 | 500.0 | 6.6 | 118.7 | 0.132 | 0.100 |
| example(8) | P-8 | 5.5 | 7.7 | 500.0 | 6.5 | 118.4 | 0.130 | 0.100 |
| example(9) | P-11 | 5.5 | 7.6 | 500.0 | 6.6 | 114.4 | 0.130 | 0.100 |
| example(10) | P-12 | 5.4 | 7.7 | 500.0 | 6.5 | 115.1 | 0.132 | 0.100 |
| example(11) | P-14 | 5.4 | 7.6 | 500.0 | 6.6 | 113.0 | 0.133 | 0.100 |
| example(12) | P-16 | 5.5 | 7.5 | 500.0 | 6.7 | 114.6 | 0.133 | 0.100 |
| example(13) | P-18 | 5.5 | 7.5 | 500.0 | 6.7 | 114.8 | 0.133 | 0.100 |
| example(14) | P-21 | 5.3 | 7.5 | 500.0 | 6.7 | 113.5 | 0.132 | 0.100 |
| example(15) | P-23 | 5.5 | 7.8 | 500.0 | 6.4 | 114.5 | 0.131 | 0.100 |
| example(16) | P-24 | 5.4 | 7.5 | 500.0 | 6.6 | 115.6 | 0.130 | 0.100 |
| example(17) | P-26 | 5.5 | 7.5 | 500.0 | 6.7 | 113.8 | 0.132 | 0.100 |
| example(18) | P-28 | 5.5 | 7.6 | 500.0 | 6.5 | 114.5 | 0.131 | 0.100 |
| example(19) | P-35 | 5.3 | 7.8 | 500.0 | 6.4 | 115.8 | 0.130 | 0.100 |
| example(20) | P-38 | 5.4 | 7.5 | 500.0 | 6.6 | 115.2 | 0.131 | 0.100 |

As can be seen from the results in Table 2, it can be seen that when a blue organic light emitting device is manufactured by using the material for an organic electroluminescent device of the present invention as an emitting auxiliary layer material, the lifespan of the organic electroluminescent device can be significantly improved compared to Comparative Examples using Comparative Compounds 1 or Comparative Compound 2. In detail, compared to Comparative Compound 1 in which was not substituted with deuterium, Comparative Compound 2, in which 45.7% of the total hydrogen was substituted with deuterium, showed improved device results with improved driving voltage, efficiency, and lifespan, and compared to Comparative Compound 2, the compound of the present invention in which 59% to 73% of the total hydrogen was substituted with deuterium showed excellent device results in terms of lifespan.

When deuterium is substituted, as the bond length of deuterium-carbon becomes shorter than that of hydrogen-carbon, the molecular hardcore volume is reduced, and thus electrical polarizability can be reduced. For this reason, the effect of lowering the crystallinity of the thin film, that is, an amorphous state can be made, and consequently, hole mobility can be increased.

It can be seen that, in particular, the compound of the present invention deuterated at a substitution rate of 59% to 73%, which is higher than the existing substitution rate, increases the BDE (Bond Dissociation Energy) compared to the comparative compound, thereby maximizing the bond stability of the structure, and as a result, the stability of the molecules in the device is improved, so that the results are remarkably excellent in terms of lifespan.

This suggests that even though they have a similar structure, the physical properties, and properties of the compound, and results of the device may be significantly different depending on the substitution rate of deuterium.

In the case of the emitting auxiliary layer, it is necessary to understand the correlation between the hole transport layer and the emitting layer (host), and even if a similar core is used, it will be very difficult for a person skilled in the art to infer the characteristics exhibited by the emitting auxiliary layer in which the compound of the present invention is used.

In addition, in the evaluation results of the above-described device fabrication, the device characteristics in which the compound of the present invention is applied only to the emitting auxiliary layer has been described, but the compound of the present invention may be applied to the hole transport layer or both the hole transport layer and the emitting auxiliary layer may be applied.

Although exemplary embodiments of the present invention have been described for illustrative purposes, those skilled in the art will appreciate that various modifications, additions and substitutions are possible, without departing from the scope and spirit of the invention as disclosed in the accompanying claims. Therefore, the embodiment disclosed in the present invention is intended to illustrate the scope of the technical idea of the present invention, and the scope of the present invention is not limited by the embodiment. The scope of the present invention shall be construed on the basis of the accompanying claims, and it shall be construed that all of the technical ideas included within the scope equivalent to the claims belong to the present invention.

### INDUSTRIAL APPLICABILITY

According to the present invention, it is possible to manufacture an organic device having excellent device characteristics of high luminance, high light emission and long life, and thus has industrial applicability.

1. A 59% to 73% deuterated compound represented by Formula 1: 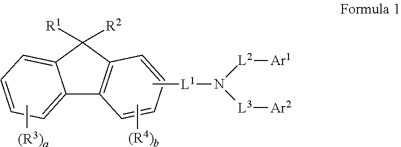
wherein:
1) R¹ and R² are each independently a C₁-C₅₀ alkyl group, and R¹ and R² do not bond to each other to form a ring,
2) R³ and R⁴ are each independently selected from the group consisting of hydrogen; deuterium; halogen; a cyano group; a nitro group; a C₆-C₆₀ aryl group; a fluorenyl group; a C₂-C₆₀ heterocyclic group including at least one heteroatom of O, N, S, Si or P; a fused ring group of a C₃-C₆₀ aliphatic ring and a C₆-C₆₀ aromatic ring; a C₁-C₅₀ alkyl group; a C₂-C₂₀ alkenyl group; a C₂-C₂₀ alkynyl group; a C₁-C₃₀ alkoxyl group; a C₆-C₃₀ aryloxy group; and -L′-N(Rₐ)(R_{b}); or, when a and b are 2 or more, a plurality of adjacent R³ or a plurality of R⁴ may be bonded to each other to form a ring,
wherein L′ is selected from the group consisting of a single bond; a C₆-C₆₀ arylene group; a fluorenylene group; a fused ring group of a C₃-C₆₀ aliphatic ring and a C₆-C₆₀ aromatic ring; a C₂-C₆₀ heterocyclic group, and Rₐ and R_{b} are each independently selected from the group consisting of a C₆-C₆₀ aryl group; a fluorenyl group; a fused ring group of a C₃-C₆₀ aliphatic ring and a C₆-C₆₀ aromatic ring; a C₂-C₆₀ heterocyclic group including at least one heteroatom of O, N, S, Si or P,
3) L¹, L² and L³ are each independently selected from the group consisting of a single bond; a C₆-C₆₀ arylene group; a fluorenylene group; a fused ring group of a C₃-C₆₀ aliphatic ring and a C₆-C₆₀ aromatic ring; and a C₂-C₆₀ heterocyclic group,
4) a is an integer of 0 to 4, b is an integer of 0 to 3,
5) Ar¹ and Ar² are each independently selected from the group consisting of a C₆-C₆₀ aryl group; a C₂-C₆₀ heterocyclic group including at least one heteroatom of O, N, S, Si or P; a fused ring group of a C₃-C₆₀ aliphatic ring and a C₆-C₆₀ aromatic ring; a C₁-C₆₀ alkyl group; a C₂-C₂₀ alkenyl group; a C₂-C₂₀ alkynyl group; a C₁-C₃₀ alkoxyl group; a C₆-C₃₀ aryloxy group; and -L′-N(Rₐ)(R_{b}),
6) wherein the aryl group, arylene group, heterocyclic group, fluorenyl group, fluorenylene group, fused ring group, alkyl group, alkenyl group, alkoxy group and aryloxy group may be substituted with one or more substituents selected from the group consisting of deuterium; halogen; a silane group; a siloxane group; a boron group; a germanium group; a cyano group; a nitro group; a C₁-C₂₀ alkylthio group; a C₁-C₂₀ alkoxyl group; a C₁-C₂₀ alkyl group; a C₂-C₂₀ alkenyl group; a C₂-C₂₀ alkynyl group; a C₆-C₂₀ aryl group; a C₆-C₂₀ aryl group substituted with deuterium; a fluorenyl group; a C₂-C₂₀ heterocyclic group; a C₃-C₂₀ cycloalkyl group; a C₇-C₂₀ arylalkyl group; and a C₈-C₂₀ arylalkenyl group; and -L′-N(Rₐ)(R_{b}), wherein the substituents may be bonded to each other to form a saturated or unsaturated ring, wherein the term ‘ring’ means a C₃-C₆₀ aliphatic ring or a C₆-C₆₀ aromatic ring or a C₂-C₆₀ heterocyclic group or a fused ring formed by the combination thereof.

2. The compound of claim 1, wherein Formula 1 is represented by any one of Formulas 2 to 4: 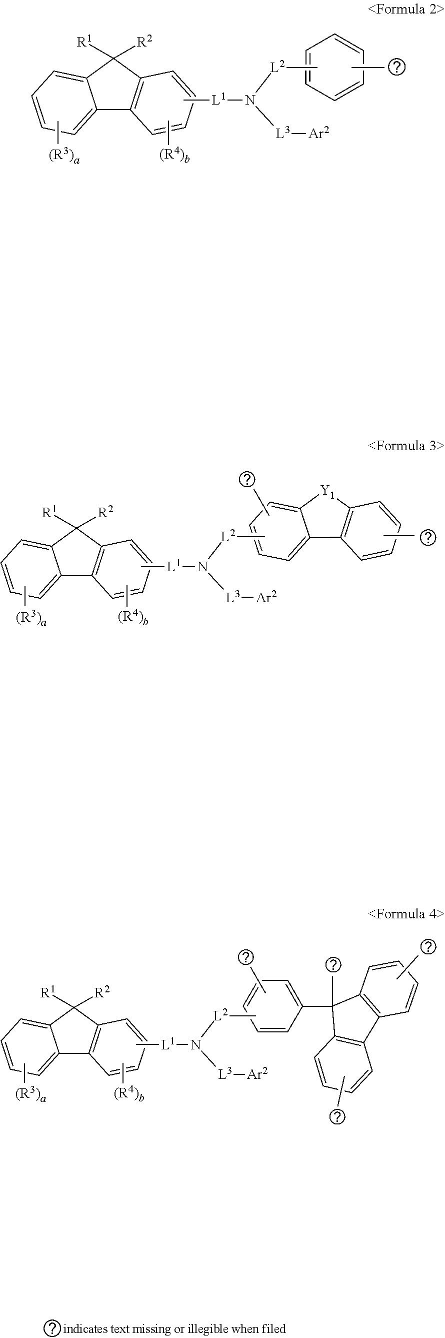
wherein:
1) R¹, R², R³, R⁴, L¹, L², L³, Ar², a and b are the same as defined in claim 1,
2) R⁵, R⁶, R⁷, R⁸ and R⁹ are the same as the definition of R³ in claim 1,
3) c is an integer of 0 to 5,
d is an integer of 0 to 3,
e, f and g are each independently an integer of 0 to 4,
4) R^{a} is selected from the group consisting of a C₁-C₅₀ alkyl group; a C₆-C₆₀ aryl group; fluorenyl group; and a C₂-C₆₀ heterocyclic group including at least one heteroatom of O, N, S, Si or P,
5) Y₁ is O, S or CR′R″,
6) wherein R′ and R″ are each independently selected from the group consisting of a C₆-C₆₀ aryl group; fluorenyl group; a C₂-C₆₀ heterocyclic group including at least one heteroatom of O, N, S, Si or P; a fused ring group of a C₃-C₆₀ aliphatic ring and a C₆-C₆₀ aromatic ring; and -L′-N(Rₐ)(R_{b}); or R′ and R″ are bonded to each other to form a C₆-C₆₀ aromatic ring; fluorenyl group; a C₂-C₆₀ heterocyclic group including at least one heteroatom of O, N, S, Si or P; a C₃-C₆₀ aliphatic ring; or a fused ring group of a C₃-C₆₀ aliphatic ring and a C₆-C₆₀ aromatic ring; and
7. wherein L′, Rₐ and R_{b} are the same as defined in claim 1.

3. The compound of claim 1, wherein Formula 1 is represented by any one of Formulas 5 to 9: 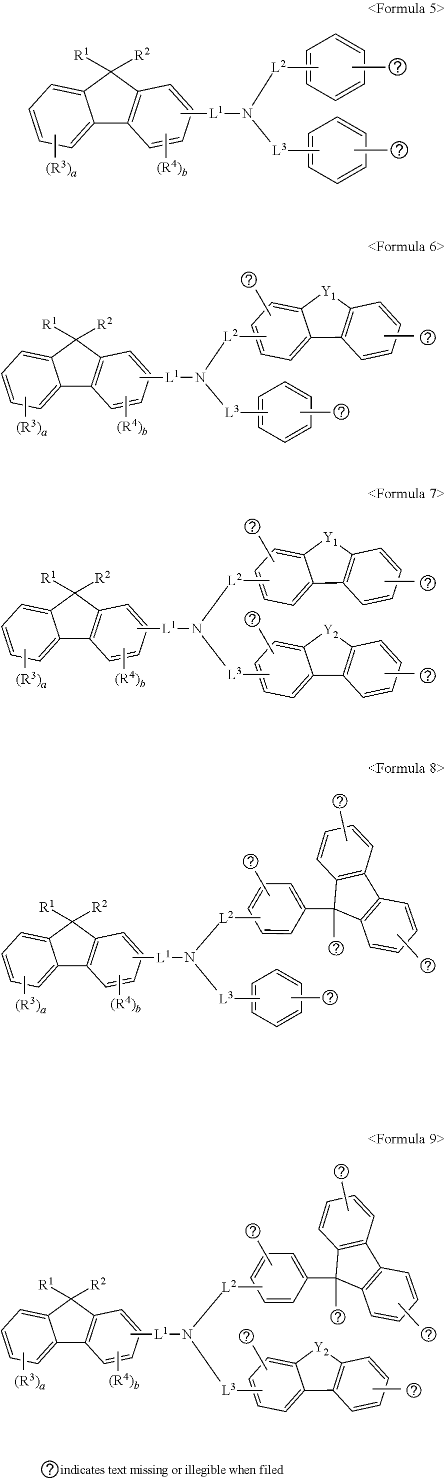
Wherein:
1) R¹, R², R³, R⁴, L¹, L², L³, a and b are the same as defined in claim 1,
2) R⁵, R⁶, R⁷, R⁸, R⁹ are the same as the definition of R³ in claim 1,
c is an integer of 0 to 5, d is an integer of 0 to 3, e, f and g are each independently an integer of 0 to 4, and Y₁ is O, S or CR′R″,
3) R^{1′}, R^{2′}and R^{3′} are the same as the definition of R³ in claim 1,
4) a′ is an integer of 0 to 5, b′ is an integer of 0 to 3, c′ is an integer of 0 to 4,
5) Y₂ is O or S.

4. The compound of claim 1, wherein Formula 1 is represented by any one of the following compounds P-1 to P-42:

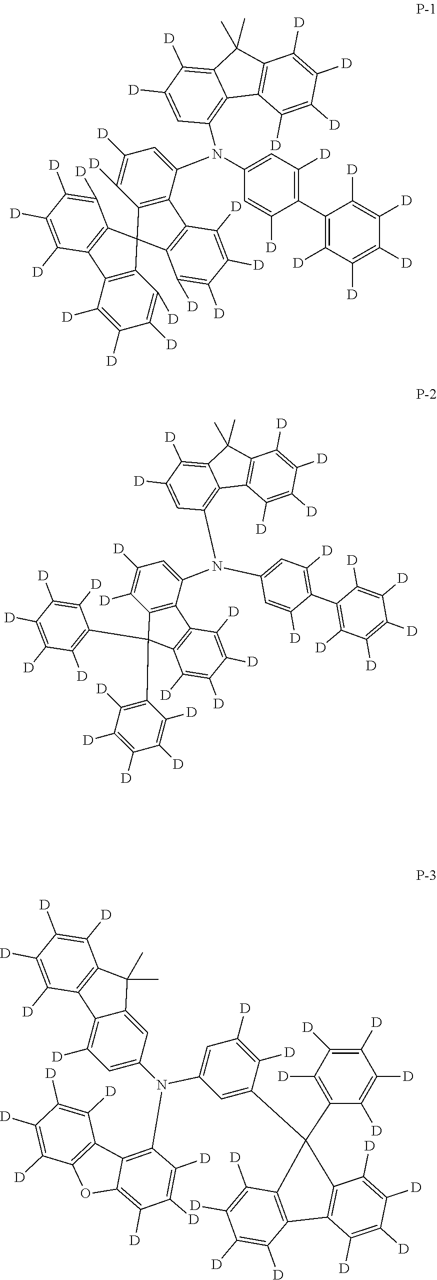

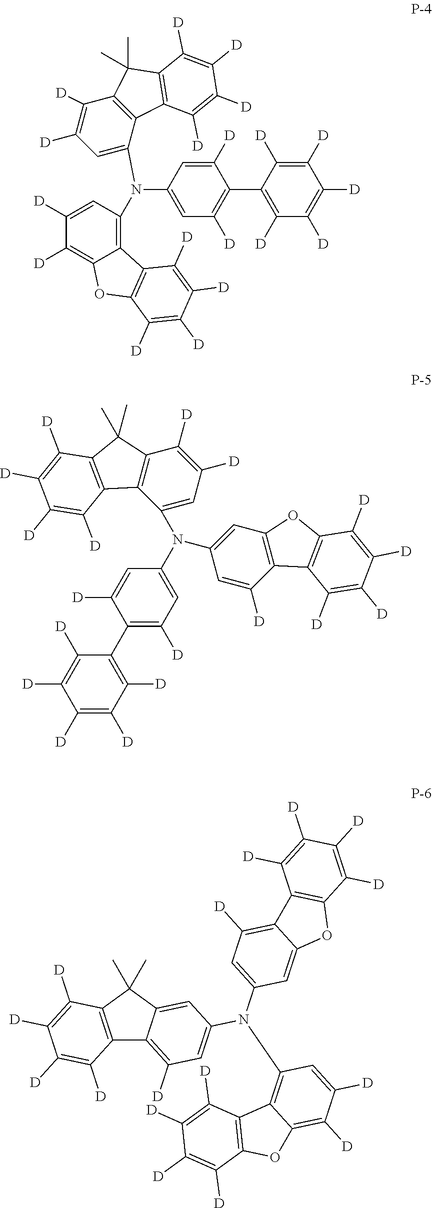

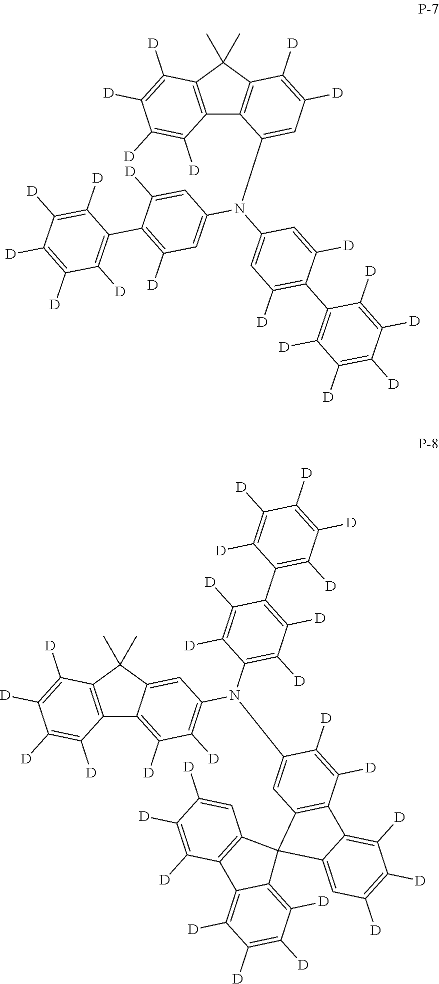 [SMILES: [2H]c1cc(N(c2cc([2H])c(-c3c([2H])c([2H])c([2H])c([2H])c3[2H])c([2H])c2)c2cc([2H])c(-c3c([2H])c([2H])c([2H])c([2H])c3[2H])c([2H])c2)c2c(c1[2H])C(C)(C)c1c([2H])c([2H])c([2H])c([2H])c1-2 ; [2H]c1cc2c(c([2H])c1[2H])-c1c(cc(N(c3cc4c(c([2H])c3[2H])-c3c([2H])c([2H])c([2H])c([2H])c3C4(C)C)c3c([2H])c([2H])c(-c4c([2H])c([2H])c([2H])c([2H])c4[2H])c([2H])c3[2H])c([2H])c1[2H])C21c2cc([2H])c([2H])c([2H])c2-c2c([2H])c([2H])c([2H])c([2H])c21]

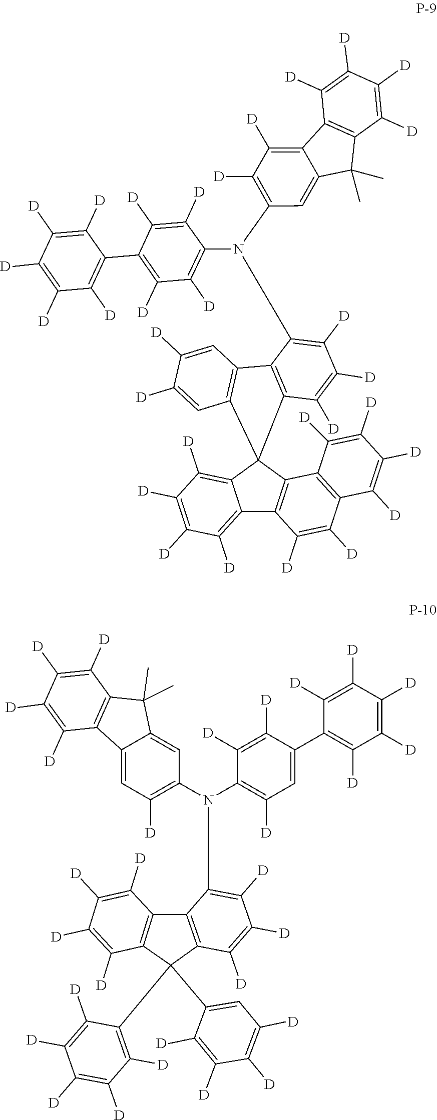

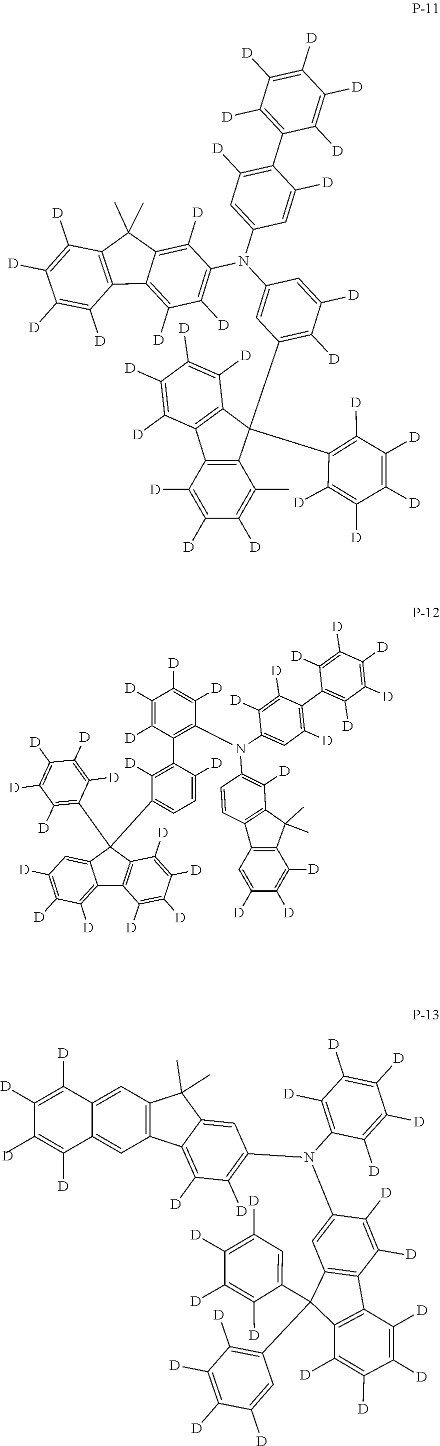

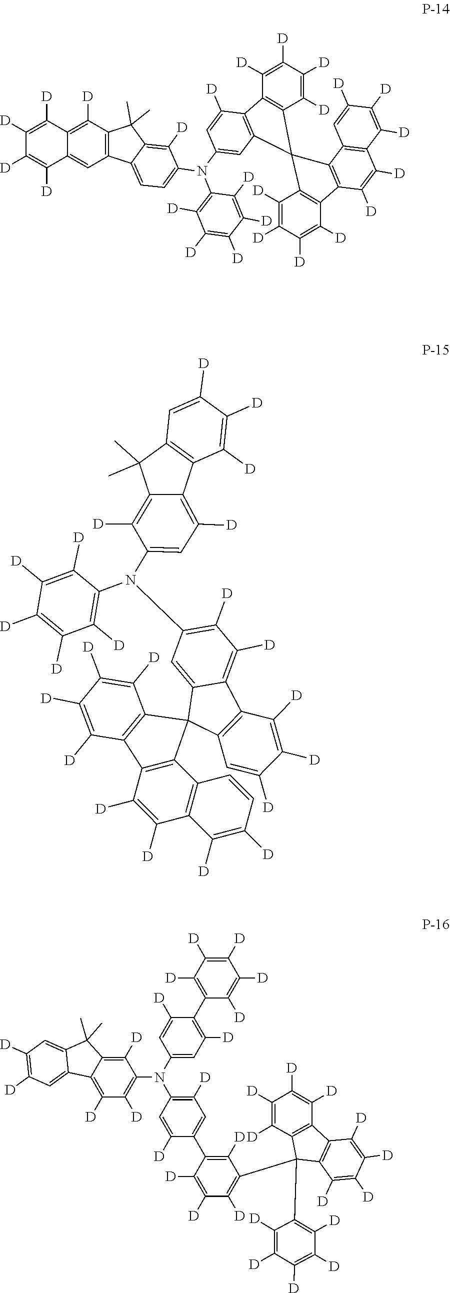

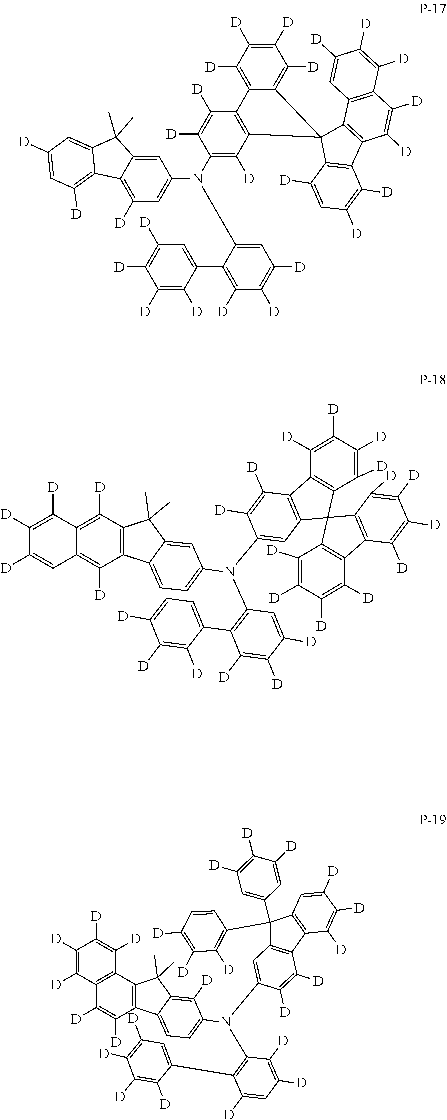

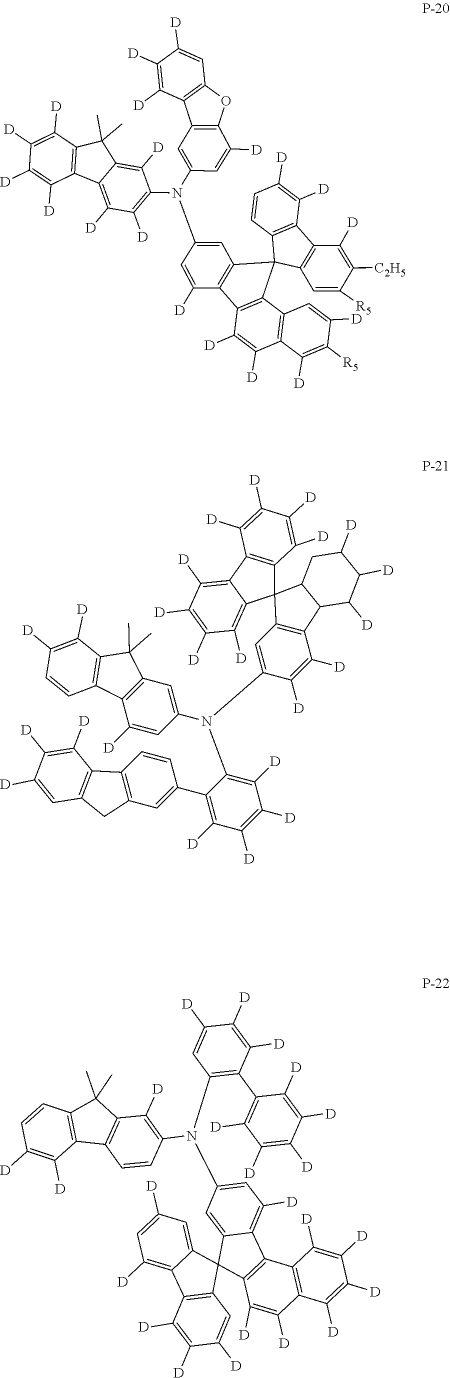

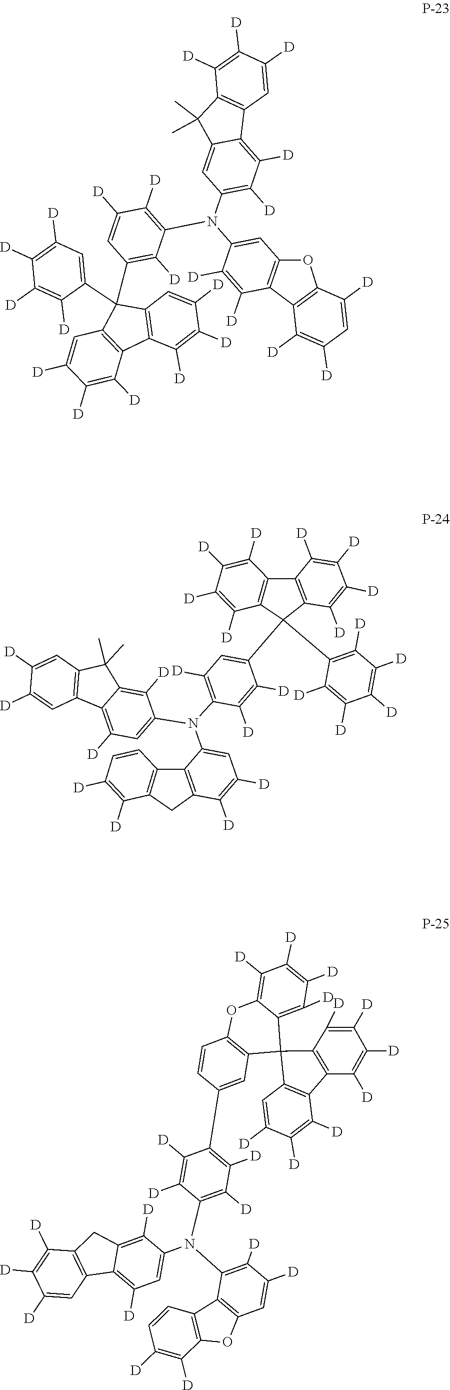

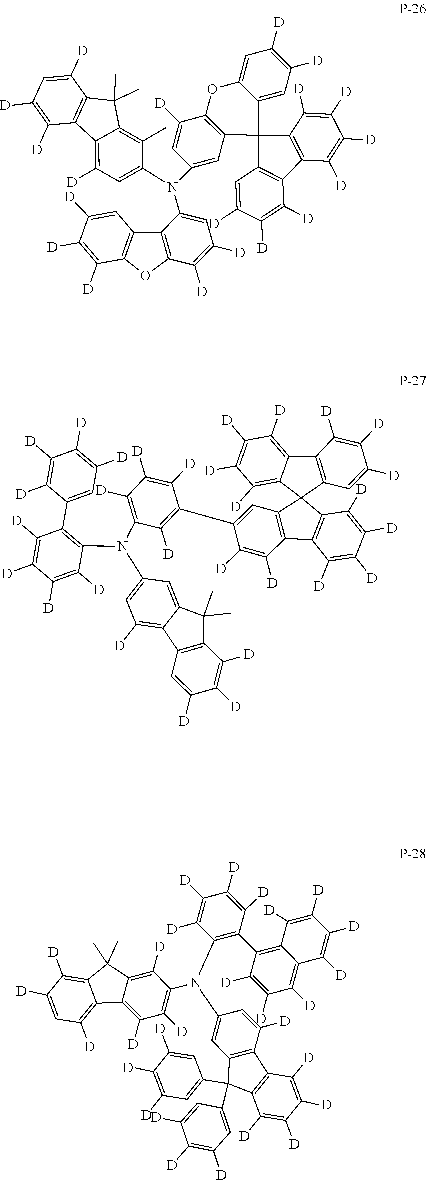

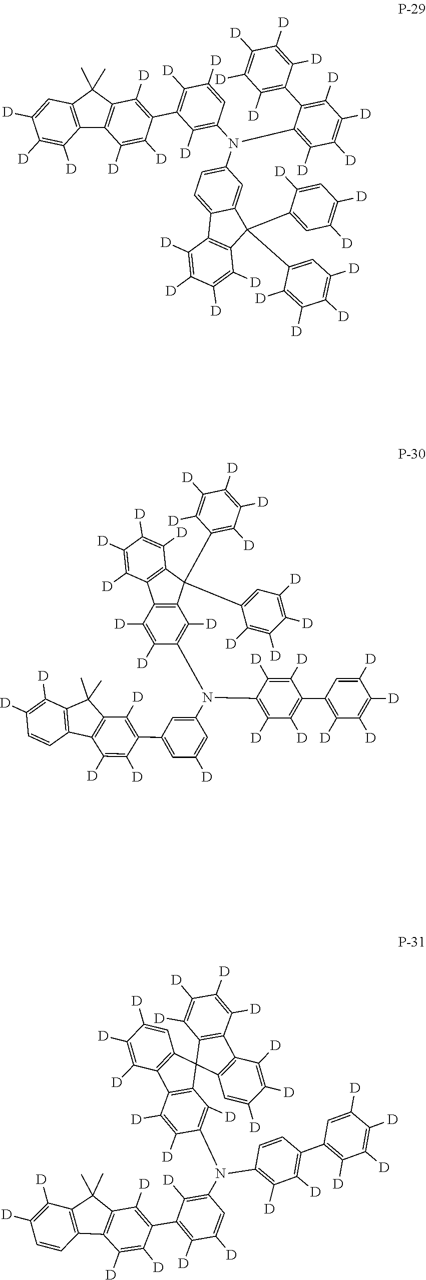

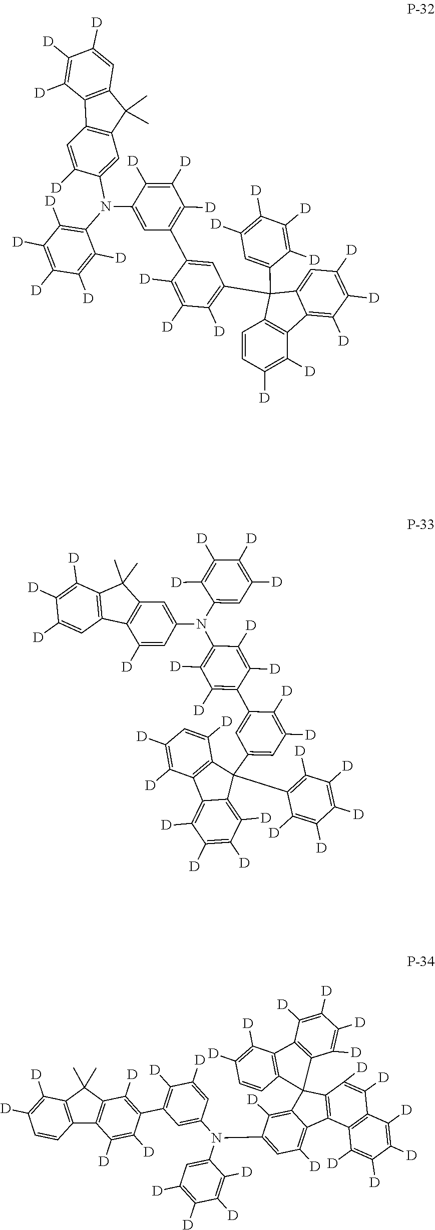

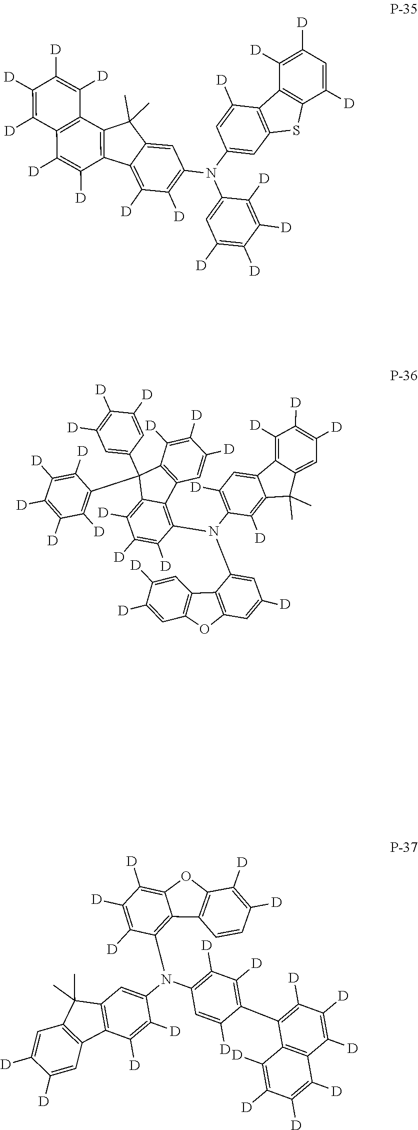

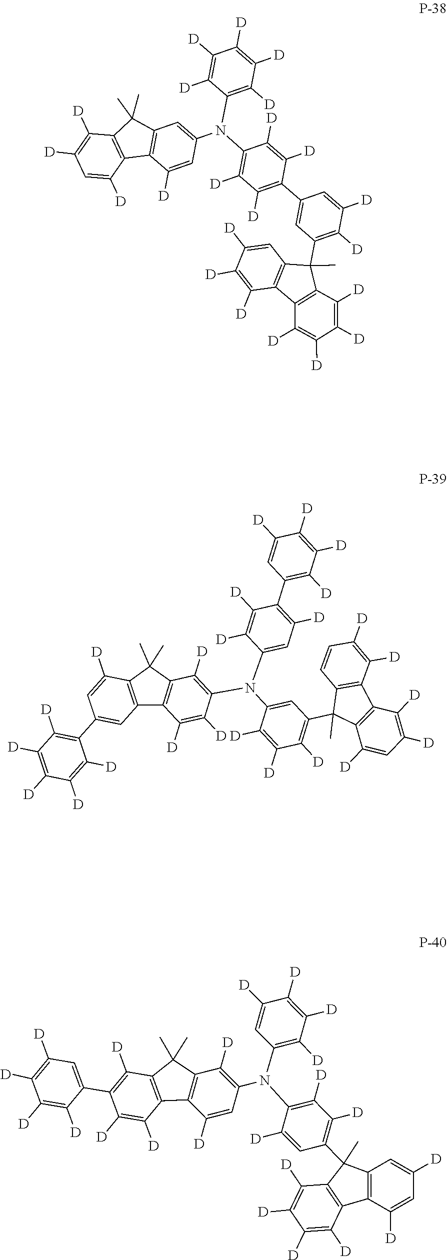

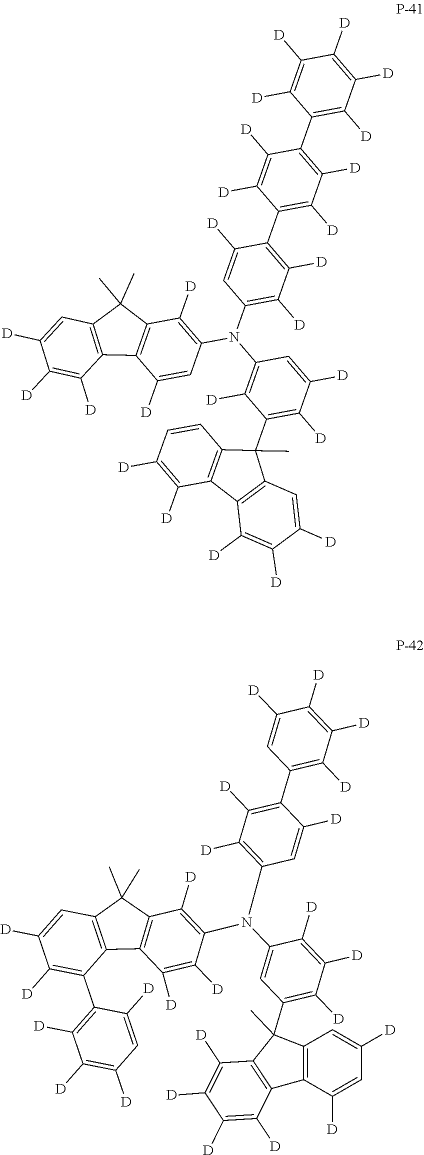 [SMILES: [2H]c1cc2c(c([2H])c1[2H])-c1c([2H])cc(N(c3cc([2H])c(-c4c([2H])c([2H])c(-c5c([2H])c([2H])c([2H])c([2H])c5[2H])c([2H])c4[2H])c([2H])c3[2H])c3cc([2H])c([2H])c(C4(C)c5ccc([2H])c([2H])c5-c5c4cc([2H])c([2H])c5[2H])c3[2H])c([2H])c1C2(C)C ; [2H]c1cc([2H])c2c(c1)C(C)(c1cc(N(c3cc([2H])c(-c4cc([2H])c([2H])c([2H])c4[2H])c([2H])c3[2H])c3c([2H])c([2H])c4c(c3[2H])C(C)(C)c3cc([2H])c([2H])c(-c5c([2H])cc([2H])c([2H])c5[2H])c3-4)c([2H])c([2H])c1[2H])c1c([2H])c([2H])c([2H])c([2H])c1-2]

5. A method for preparing the 59%˜73% deuterated compound represented by Formula 1 according to claim 1 comprising:
(a) a step of forming a first reactant by dissolving the compound represented by Formula 1 in perdeuterated benzene (benzene-D₆);
(b) a step of forming a second reactant by adding deuterium-triflic acid (CF₃SO₃D) to the first reactant;
(c) a step of deuterating the second reactant by reacting the second reactant at 80° C. for 3 hours to 18 hours;
(d) a step of quenching by adding Na₂CO₃ in D₂O after the reaction in step (c) is completed and the second reactant is cooled to room temperature,
(e) a step of obtaining the deuterated compound represented by Formula 1 after concentrating the organic solvent of the second reactant and recrystallizing with toluene and acetone solvent.

6. An organic electric element comprising an anode, a cathode, and an organic material layer formed between the anode and the cathode, wherein the organic material layer comprises a single compound or 2 or more compounds represented by Formula 1 of claim 1.

7. The organic electric element of claim 6, wherein the organic material layer comprises at least one of a hole injection layer, a hole transport layer, an emitting auxiliary layer, an emitting layer, an electron transport auxiliary layer, an electron transport layer, and an electron injection layer.

8. The organic electric element of claim 6, wherein the organic material layer is an emitting auxiliary layer.

9. The organic electric element of claim 6, wherein the organic material layer is a hole transport layer.

10. The organic electric element of claim 6, wherein the organic electric element further comprises a light efficiency enhancing layer formed on at least one surface of the anode and the cathode, the surface being opposite to the organic material layer.

11. The organic electric element of claim 6, wherein the organic material layer comprises 2 or more stacks including a hole transport layer, an emitting layer, and an electron transport layer sequentially formed on the anode.

12. The organic electric element of claim 6, wherein the organic material layer further comprises a charge generation layer formed between the 2 or more stacks.

13. An electronic device comprising a display device comprising the organic electric element of claim 6; and a control unit for driving the display device.

14. An electronic device according to claim 13, wherein the organic electronic element is at least one of an OLED, an organic solar cell, an organic photo conductor(OPC), organic transistor (organic TFT) and an element for monochromic or white illumination.
